(19) United States
(12) Patent Application Publication
(10) Pub. No.: US 2006/0018921 A1
(43) Pub. Date: Jan. 26, 2006

(54) Histone deacetylase inhibitors and cognitive applications

(75) Inventors: Jonathan M. Levenson, Houston, TX (US); J. David Sweatt, Houston, TX (US)

Correspondence Address: Fulbright & Jaworski L.L.P.;Fulbright Tower, Suite 5100, 1301 McKinney, Houston, TX, 77010-3095, US

(73) Assignee: BAYLOR COLLEGE OF MEDICINE, Houston, TX (US)

(21) Appl. No.: 11/177,824

(22) Filed: Jul. 8, 2005

Related U.S. Application Data
(60) Provisional application No. 60/588,443, filed on Jul. 16, 2004.

(51) Int. Cl. A61K 38/12; A61K 39/002; A61K 31/19

(52) U.S. Cl. 424/191.1; 514/9; 514/575; 514/557

(57) ABSTRACT

The present invention relates to the enhancement of cognition in an individual by delivery of a histone acetylation regulator, such as a histone deacetylase inhibitor. The individual may have normal or poor memory, and the poor memory may be the result of a pathogenic condition or a non-pathogenic condition, such as with normal aging-related impairment. In a specific embodiment, enhancement of cognition occurs in an individual having a mental retardation syndrome.

The present invention claims priority to U.S. Provisional Patent Application Ser. No. 60/588,443, filed Jul. 16, 2004, which is incorporated by reference herein in its entirety.

### STATEMENT REGARDING FEDERALLY SPONSORED RESEARCH OR DEVELOPMENT

The present invention utilized funds from NIMH grant number MH-57014. The United States Government may have certain rights in the invention.

### TECHNICAL FIELD OF THE INVENTION

The present invention generally regards the fields of cellular biology, molecular biology, and medicine. Specifically, the present invention regards enhancement of cognition, such as memory, with histone acetylation regulators.

### BACKGROUND OF THE INVENTION

Approaches to cognitive enhancement for individuals are desirable, particularly for those suffering from cognitive impairments, including memory enhancement and impairment. Early in the study of memory, it was discovered that transcription is required for the formation of Long-term Memory (LTM) (Appel, 1965; Nakajima, 1972; Codish, 1971; Nakajima, 1969). Moreover, during the last 2 decades, several genes have been identified whose transcription is regulated during memory formation (Hall et al., 2000; Cole et al., 1989; Leil et al., 2003; Levenson et al., 2004). Current hypotheses reasonably posit that the activity of many transcription factors, shown to increase in phosphorylation or protein level, during periods of memory formation trigger the changes in gene expression necessary for the consolidation of long-term memory (Levenson et al., 2004; Jones et al., 2001; Taubenfeld et al., 2001; Sananbenesi et al., 2002; Fleischmann et al., 2003; Barco et al., 2002; Bourtchuladze et al., 1994; Impey et al., 1998; Davis et al., 2000; Meffert et al., 2003). However, the present invention addresses if mechanisms aside from direct transcription factor activation might contribute to memory consolidation and activity-dependent plasticity. Specifically, the present invention addresses alterations in chromatin structure contributing to memory-associated regulation of gene expression.

In the nucleus, DNA is tightly packaged into chromatin, a DNA-protein complex (Varga-Weisz and Becker, 1998). The major protein component of chromatin comprises a group of highly basic proteins known as histones (Varga-Weisz and Becker, 1998). The basic functional unit of chromatin is the nucleosome; each nucleosome is a histone octamer made up of a tetramer formed from 2 histone H3-H4 dimers, and 2 additional histone H2A-H2B dimers (Varga-Weisz and Becker, 1998). Each nucleosome accommodates approximately 146 bp of DNA wrapped around the histone octet 1.67 times (Varga-Weisz and Becker, 1998). There is 20-50 bp of “linker” DNA between each nucleosome that interacts with 1 molecule of histone H1 (Varga-Weisz and Becker, 1998). Positively charged lysine residues in the N-terminal tail of histones mediate most of the DNA-histone interaction. Unmodified, native histones tightly bind to DNA, preventing other protein interactions including the RNA polymerase II enzyme (RNApolII) interaction required for transcription (Varga-Weisz and Becker, 1998). Thus, in its native state histone-associated heterochromatin is highly inhibitory to transcription (Varga-Weisz and Becker, 1998).

Transcription can be divided into 2 phases: initiation and elongation (Varga-Weisz and Becker, 1998; Orphanides and Reinberg, 2000). For either stage of transcription to occur, the native structure of chromatin must be disrupted. Acetylation of the ε-amino group of lysine residues by histone acetyltransferases (HATs) neutralizes the positive charge of histones, disrupting the interaction between histone and DNA (Varga-Weisz and Becker, 1998). Some HATs have been shown to preferentially acetylate histones on specific lysine residues. For example, the HATs HPA2 and Gcn5 appear to preferentially acetylate histone H3 on Lys14, a residue that has been implicated in regulation of transcription (Angus-Hill et al., 1999; Cheung et al., 2000). Histone acetylation appears to increase initiation by facilitating the binding of transcription factors and the RNA polII holoenzyme to DNA (Orphanides and Reinberg, 2000; Freedman, 1999). It should also be noted that acetylation of histones can in some cases be self-perpetuating, which in development may serve as a long-term cellular memory, creating a functionally stable chromatin state and thus chronic changes in the rates of specific gene expression (Turner, 2002).

Several studies have suggested that regulation of histones, and thus chromatin structure, is an important step in modulating transcription and facilitating long-term changes in neuronal physiology. Gross changes in chromatin structure have been demonstrated in the suprachiasmatic nucleus when animals are exposed to phase-resetting light pulses, and chromatin structure is regulated in hippocampal neurons in response to activation of a variety of neurotransmitter pathways (Crosio et al., 2000; Crosio et al., 2003). Some studies have also examined the structure of chromatin within specific gene-associated promoters. Status epilepticus induces changes in the structure of chromatin associated with the GluR2 and BDNF genes in area CA3 (Huang et al., 2002). Other studies have found regulation of chromatin structure around the ApC/EBP gene in Aplysia using a pharmacologic paradigm that triggers long-term facilitation of neurotransmitter release at the sensory-motor neuron synapse in this species (Guan et al., 2002; Guan et al., 2003).

Chang et al. (2001) examined the effects of HDAC inhibitors on spinal muscular atrophy (SMA). This study showed that sodium butyrate was effective at increasing the amount of exon 7-containing survival motor neuron (SMN) protein in SMA lymphoid cell lines by changing the alternative splice pattern of exon 7 in the SMN2 gene, which has been shown to be protective from SMA effects. In vivo, SMA-like mice showed increased expression of SMN protein in spinal cord motor neurons when treated with sodium butyrate, as well as improved symptoms. The drug also decreased the birth rate of severe types of SMA-like mice when heterozygous knock-out transgenic SMA-like mice were bred.

Steffan et al. (2001) explored the ability of HDAC inhibitors to affect Huntington's Disease. There, it was shown that HDAC inhibitors could reverse the reduction in acetylated H3 and H4 histones caused by Httex1p in cell free assays, a protein that binds the acetyltransferase domains of CREB-binding protein and p300. In vivo, HDAC inhibitors arrest ongoing progressive neuronal degeneration induced by polyglutamine repeat expansion, and reduce lethality in two Drosophila models of polyglutamine disease, suggesting a potential role in treatment of Huntington's Disease and other polyglutamine-repeat diseases.

In two recent studies by Korzus et al. (2004) and Alarcon et al. (2004), mutant animals were developed that had impaired CREB-binding protein (CBP) activity. These CBP mutants were a model for Rubinstein Taybi Syndrome, a form of mental retardation that is due to deficiencies in CBP activity (Petrij, 1995). In the studies by Korzus et al. and Alarcon et al., it was shown that infusion of the histone deacetylase inhibitor Trichostatin A directly into the brain of animals deficient in CBP improved the animals' performance in some long-term memory tasks. However, the authors did not assess effects of HDAC inhibitors on normal or aging-related memory and cognition.

U.S. 20040077591 relates to histone deacetylase inhibitors for the treatment of Alzheimer's Disease, multiple sclerosis, and amyotrophic lateral sclerosis.

Thus, the related art lacks a system, methods, and/or compositions for enhancing cognition, and particularly memory, that the novel present invention provides.

### BRIEF SUMMARY OF THE INVENTION

The present invention is directed to a system, method, and compositions that augment cognition, treat a medical condition associated with memory decline or decreased mental capacity (such as a learning disability), and/or improve memory in individuals with normal or poor memory. These embodiments are associated with changes in chromatin structure and/or function, particularly via histone acetylation modification. In specific aspects of the invention, chromatin structure is highly dynamic within the nervous system, and mechanisms and compositions of the present invention are recruited as a target of plasticity-associated signal transduction mechanisms.

Regulators of histone acetylation, such as histone deacetylase (HDAC) inhibitors, are included in the invention. In a specific embodiment of the present invention, there are provided methods and compositions regarding regulation of histone acetylation during memory formation in the hippocampus.

Formation of long-term memory begins with the activation of many disparate signaling pathways that ultimately impinge on the cellular mechanisms regulating gene expression. In the present invention, the structure of chromatin was examined, as assessed by histone acetylation, particularly during induction of long-term memory formation, such as in the hippocampus, and for example in area CA1 of the hippocampus. Contextual fear conditioning increased acetylation of histone H3, but not H4, although in alternative embodiments acetylation of H4 is associated with the present invention. Furthermore, stimulation of a known memory-associated signaling pathway, ERK, through activation of either the protein kinase C (PKC) or protein kinase A (PKA) pathways, also increased acetylation of histone H3 without affecting H4. Finally, the present inventors found that artificially elevating levels of histone acetylation in vitro, such as through HDAC inhibitors, enhances the induction of a lasting form of synaptic strengthening, long-term potentiation (LTP) in hippocampal area CA1. Taken together these observations indicate that there are alterations in histone acetylation, and by extension modulation of chromatin structure, in mammalian associative learning and long-term memory formation, for example.

Particular but exemplary embodiments of medical conditions treatable by the present invention include, for example, mild cognitive impairment (MCI); aging-related memory impairment; non-syndromic mental retardation; Rett syndrome; fragile X mental retardation; Down syndrome; attention deficit disorder; developmental delay; Angelman syndrome; cognitive enhancement in low-IQ individuals; and Rubinstein-Taybi Syndrome.

In one embodiment of the present invention, there is a method of enhancing cognition in an individual, comprising the step of delivering to the individual an effective amount of a histone acetylation regulator. In specific embodiments, the individual comprises a medical condition selected from the group consisting of mild cognitive impairment (MCI), aging-related memory impairment, non-syndromic mental retardation, Rett syndrome, fragile X mental retardation, Down syndrome, attention deficit disorder, developmental delay, or Angelman syndrome. In a specific embodiment, the histone acetylation regulator comprises a histone acetyltransferase activator or activity enhancer. In another specific embodiment, the histone acetylation regulator comprises a histone deacetylase inhibitor, such as trichostatin A, trichostatin B, trichostatin C, trapoxin A, trapoxin B, chlamydocin, sodium butyrate, sodium phenylbutyrate, MS-27-275, scriptaid, FR901228, depudecin, oxamflatin, pyroxamide, apicidin B, apicidin C, Helminthsporium carbonum toxin, 2-amino-8-oxo-9,10-epoxy-decanoyl, 3-(4-aroyl-1H-pyrrol-2-yl)-N-hydroxy-2-propenamide, suberoylanilide hydroxamic acid, FK228, m-carboxycinnamic acid bis-hydroxamide, or a mixture thereof.

In specific aspects of the invention, methods and compositions are further defined as being directed to enhancing memory in an individual, such as, for example, long-term memory in the individual. The individual may comprise substantially normal memory faculty or substantially sub-normal memory faculty. In specific embodiments, the sub-normal memory faculty results from a pathogenic condition, such as one comprising a mental retardation syndrome, mild cognitive impairment (MCI), attention deficit disorder, developmental delay, aging-related memory impairment, cognitive enhancement in low-IQ individuals, non-syndromic mental retardation, bipolar disorder, head trauma, seizures, alcoholism, stroke, transient ischemic attack (TIA), transient global amnesia, electroconvulsive therapy, brain mass, infection, depression, AIDS-related cognitive impairment, or a combination thereof. Exemplary mental retardation syndromes include Rett syndrome, fragile X mental retardation, Down syndrome, Angelman syndrome.

In another specific embodiment, the sub-normal memory faculty results from a non-pathogenic condition, such as one comprising anesthesia, an illicit/illegal drug, temporal lobe brain surgery, or a combination thereof. Exemplary anesthetics comprise halothane, isoflurane, fentanyl, or a mixture thereof. Exemplary illicit/illegal drugs comprise barbiturates, benzodiazepines, marijuana, cocaine, heroin, methamphetamine, inhalants, Ecstasy, GHB, ketamine or a mixture thereof. In a specific embodiment, the non-pathogenic condition comprises normal age-related impairment, such as one comprising mild cognitive impairment (MCI).

In a particular embodiment, the method is further defined as reducing the activity of a nuclear memory repressor. In another particular embodiment, the method does not substantially affect basal synaptic transmission, short-term plasticity, or both.

In an additional embodiment of the present invention, there is a method of improving memory in an individual having a disease associated therewith, comprising the step of delivering to the individual an effective amount of a histone acetylation regulator, wherein the disease comprises a mental retardation syndrome, mild cognitive impairment (MCI), attention deficit disorder, developmental delay, aging-related memory impairment, cognitive enhancement in low-IQ individuals, non-syndromic mental retardation, bipolar disorder, head trauma, seizures, alcoholism, stroke, transient ischemic attack (TIA), transient global amnesia, electroconvulsive therapy, brain mass, infection, depression, AIDS-related cognitive impairment, or a combination thereof.

In another embodiment, there is a method of treating at least one cognition-associated symptom of an individual with mental retardation, comprising the step of delivering to the individual a therapeutically effective amount of a histone acetylation regulator. The cognition-associated symptom may comprise learning disability, substantially sub-normal memory faculty, or a combination thereof. In specific embodiments, the histone acetylation regulator is a histone deacetylase inhibitor.

In particular aspects, an individual having no memory defects (or substantially no memory defects) is administered one or more methods and/or compositions of the invention. In alternative embodiments, there is no memory defect, such as there being no clinically-defined memory defect, although the individual may have a perceived cognitive deficit, including a perceived memory defect. In specific embodiments, the individual with normal memory seeks improvement of the normal memory. Such an improvement may be desired for any purpose, including for routine activities and daily life, for scholastic studies, and/or for work enhancement, for example.

The foregoing has outlined rather broadly the features and technical advantages of the present invention in order that the detailed description of the invention that follows may be better understood. Additional features and advantages of the invention will be described hereinafter which form the subject of the claims of the invention. It should be appreciated that the conception and specific embodiment disclosed may be readily utilized as a basis for modifying or designing other structures for carrying out the same purposes of the present invention. It should also be realized that such equivalent constructions do not depart from the invention as set forth in the appended claims. The novel features which are believed to be characteristic of the invention, both as to its organization and method of operation, together with further objects and advantages will be better understood from the following description when considered in connection with the accompanying figures. It is to be expressly understood, however, that each of the figures is provided for the purpose of illustration and description only and is not intended as a definition of the limits of the present invention.

### BRIEF DESCRIPTION OF THE DRAWINGS

For a more complete understanding of the present invention, reference is now made to the following descriptions taken in conjunction with the accompanying drawings.

FIGS. 1A-1D shows that contextual fear conditioning increases phosphorylation of ERK2 and acetylation of histone H3. Animals were fear conditioned, and area CA1 of the hippocampus was isolated 1 h or 24 h after training. For this and subsequent figures illustrating data collected via western blotting, representative western blots are shown above graphs of summary quantification. For each representative immunoblot, a matched pair is shown where the control sample is on the left and the experimental sample is on the right. In FIG. 1A, significant regulation of ERK2 phosphorylation was seen after contextual fear conditioning (H_{[5,31]}=25, p<0.0001). Contextual fear conditioning led to an increase in P-ERK2 1 h after fear conditioning (n=10), but not 24 h after (n=4). No regulation of ERK2 was seen after latent inhibition training (n=11). Injection of animals 1 h prior to training with the NMDA-R antagonist MK-801 (300 μg/kg, n=6) or immediately after training with the MEK inhibitor SL327 (100 mg/kg, n=6) inhibited the increase in P-ERK2. In FIG. 1B, acetylation of histones H3 and H4 was measured in area CA1 from naïve (N) and fear conditioned (FC) animals 1 h after contextual fear conditioning. Shown are representative western blots depicting levels of acetylated histones and total histone protein from acid extracts of nuclear preparations. Below the immunoblots is a diagram illustrating the various behavioral manipulations performed in our studies. In FIG. 1C, significant regulation of histone H3 acetylation was observed after contextual fear conditioning (H_{[5,31]}=15, p<0.01). Contextual fear conditioning led to an increase in the acetylation of histone H3 1 h after training (n=11), but not 24 h after (n=6). No change in H3 acetylation was observed after latent inhibition training (n=10), or injection with either MK-801 (n=9) or SL327 (n=6). In FIG. 1D, no significant regulation of histone H4 acetylation was observed 1 h or 24 h after fear conditioning (n=11). Injection of MK-801 (n=9) or SL327 (n=3) also had no effect on histone H4 acetylation. However, latent inhibition training significantly increased acetylation of histone H4 (n=11, H_{[5,30]}=10, p<0.05). Error bars indicate SEM, asterisks (*) indicate significant differences (p<0.05) as determined by post-hoc Dunn's multiple comparison test.

FIGS. 2A-2D show contextual fear memory requires proper timing and NMDA-R function. Animals were exposed to the contextual fear conditioning paradigm and freezing behavior was measured either during training (FIGS. 2A and 2C) or 24 h after training (FIGS. 2B and 2D) in the training chamber. In FIG. 2A, animals exposed to the standard contextual fear conditioning paradigm (Context+Shock, n=12) or the latent inhibition training paradigm (2 h pre-exposure to training chamber, n=12) exhibited similar amounts of freezing during the training session. In FIG. 2B, animals exposed to the contextual fear conditioning paradigm had significantly more freezing behavior than animals exposed to the latent inhibition training paradigm (t=4.7, df=20, p<0.0001). In FIG. 2C, animals injected with either saline (n=5) or MK-801 (100 μg/kg: n=3; 300 μg/kg: n=4) 1 h prior to contextual fear conditioning showed similar amounts of freezing behavior during training (F_{[2,11]}=2.4, p<0.2). In FIG. 2D, animals injected with 300 μg/kg MK-801 showed less freezing behavior than animals injected with saline 24 h after contextual fear conditioning (F_{[2,11]}=8.5, p<0.01). Error bars are SEM. Asterisks (*) indicate significant differences (p<0.05).

FIGS. 3A-3B show activation of NMDA-Rs increase phosphorylation of ERK2 and acetylation of histone H3. Acute hippocampal slices were treated with 100 μM NMDA in Mg⁺⁺-free ACSF for 10 min. After treatment, area CA1 was isolated and processed for western blotting. In FIG. 3A, phosphorylation of ERK2 was significantly increased in area CA1 after treatment with NMDA (n=5, H_{[3,10]}=9, p<0.05). The MEK inhibitors U0126 (20 μM, n=4) and PD98059 (50 μM, n=5) inhibited the affect of NMDA on P-ERK2. In FIG. 3B, acetylation of histone H3 was significantly increased in area CA1 after treatment of slices with NMDA (n=4, H_{[3,10]}=7, p<0.05). Both U0126 (n=4) and PD98059 (n=5) inhibited the affect of NMDA on acetylation of histone H3. In FIG. 3C, NMDA had no affect on acetylation of histone H4 (H_{[3,10]}=1, p<0.7). Error bars are SEM. Asterisks (*) indicate significant differences (p<0.05) as determined by post-hoc Dunn's multiple comparisons test.

FIGS. 4A-4B show that regulation of ERK phosphorylation. ERK phosphorylation was monitored in acute hippocampal slices in response to treatments with various drugs for 1 h. Significant regulation of P-ERK2 was observed (H_{[10,49]}=37, p<0.0001). In FIG. 4A, there are representative immunoblots illustrating the effect of various treatments on levels of P-ERK1/2. Blots for phosphorylated-ERK are shown on top and blots of total ERK are shown on the bottom. In each blot, control samples are on the left and experimental samples are on the right. In FIG. 4B, there is summary quantification of the regulation of P-ERK2. Treatment of slices with either PDA (3 μM, n=12) or FSK (50 μM with 100 μM Ro20-1724, n=9) led to significant increases in levels of P-ERK2. Pretreatment with either U0126 (‘U0’, 20 μM) or PD98059 (‘PD9’, 50 μM) blocked the effect of PDA (U0: n=5, PD9: n=3) and FSK (U0: n=4, PD9: n=3). Levels of P-ERK2 were diminished by treatment with either U0126 (n=6) or PD98059 (n=3) alone. The inactive analogs of PDA and FSK, 4αP (3 μM, n=3) and dFSK (50 μM with 100 μM Ro20-1724, n=3), had no effect on P-ERK2. The HDAC inhibitor TSA (n=9) had no effect on P-ERK2. Error bars indicate SEM. Asterisks (*) indicate significant differences as determined by post-hoc Dunn's multiple comparison test. TSA data were analyzed via Wilcoxon Signed Rank Test.

FIGS. 5A-5B show regulation of histone H3 acetylation by activation of ERK. Acetylation of histone H3 was monitored in acute hippocampal slices in response to treatments with various drugs for 1 h. Significant regulation of H3 acetylation was observed (H_{[10,43}]=38, p<0.0001). In FIG. 5A, there are representative immunoblots illustrating the regulation of histone H3 acetylation. Blots for acetylated histone H3 (Lys14) are shown on top while blots for total histone H3 are shown on the bottom. In FIG. 5B there is summary quantification of histone H3 acetylation. Treatment of slices with either PDA (3 μM, n=11) or FSK (50 μM with 100 μM Ro20-1724, n=11) increased histone H3 acetylation. The effects of PDA and FSK were inhibited by pretreatment with either U0126 (20 μM, PDA: n=6, FSK: n=4) or PD98059 (50 μM, PDA: n=3, FSK: n=3). The inactive analogs of PDA and FSK, 4αP (3 μM, n=3) and dFSK (50 μM with 100 μM Ro20-1724, n=3), had no effect on H3 acetylation. Inhibition of MEK using either U0126 (20 μM, n=4) or PD98059 (50 μM, n=3) had no effect on basal levels of acetylation of histone H3. The HDAC inhibitor TSA increased acetylation of histone H3 (1.65 μM, n=7). Error bars indicate SEM. Asterisks (*) indicate significant differences (p<0.05) as assessed by Dunn's multiple comparison test. TSA data were analyzed via Wilcoxon Signed Rank Test.

FIGS. 6A-6B show that acetylation of histone H4 is not regulated by activation of ERK. Acetylation of histone H4 was monitored in acute hippocampal slices in response to treatments with various drugs for 1 h. No significant regulation of H4 acetylation was observed (H_{[10,37]}=7, p<0.7). In FIG. 6A, there are representative immunoblots illustrating the regulation of histone H4 acetylation. Blots for acetylated histone H4 (Lys5/Lys8/Lys12/Lys16) are shown on top, while blots for total histone H4 protein are on the bottom. B) Summary quantification of histone H4 acetylation. TSA (1.65 μM, n=7) was the only treatment that had a significant effect on H4 acetylation. Error bars are SEM. Asterisk indicates significantly different (p<0.01) from 100% as assessed by Wilcoxon Signed Rank Test.

FIGS. 7A-7E demonstrate that trichostatin A enhanced induction of LTP. The effect of the HDAC inhibitor TSA was assessed on LTP and basal synaptic transmission of Schaffer-collateral synapses. In FIG. 7A, there is induction of L-LTP by 2, 100 Hz (1 sec, 20 sec interstimulus interval) tetani was significantly enhanced in slices treated with TSA (1.65 μM, open diamonds, n=24) relative to slices exposed to the vehicle (0.05% EtOH, closed circles, n=18; F_{[1,2,783]}=558, p<0.0001). Continuous treatment of slices with TSA where LTP was not induced had no effect on baseline synaptic efficacy for up to 3 h after the start of treatment (crosses, n=10; H_{[103,991]}=50.34, p=1.0). Representative traces 4 min before (dashed line) and 180 min after (solid line) induction of LTP are shown to the right of summary plot. Calibration bar indicates 5 msec and 1 mV. In FIG. 7B, there are input/output relationships for slices treated with vehicle or TSA for 20 min. No significant differences were seen (F_{[2,10]}=4, p<0.1). In FIG. 7C, there is paired-pulse facilitation was unaffected by treatment of slices with TSA for 20 min (F_{[1,129]}=0.01, p<1.0). In FIG. 7D there is input/output relationships for slices treated with vehicle or TSA for 3 h. No significant differences were seen (F[_{1,13]}=0.1, p<0.8). In FIG. 7E, there is paired-pulse facilitation was unaffected by treatment of slices with TSA for 3 h (F_{[1,168]}=0.8, p<0.4). These results suggest that the enhancement of LTP by TSA was not due to direct effects on synaptic transmission. In all panels, error bars are SEM.

FIGS. 8A-8C demonstrate that TSA does not affect NMDA-Rs, the NMDA-R—dependence of LTP induction, or tetanus-induced synaptic depolarization. In FIG. 8A, slices were exposed to the AMPA-R antagonist CNQX (20 μM) and perfused with an ACSF containing 4 mM CaCl₂ and no MgCl₂. The resulting field potentials represent NMDA-R mediated synaptic transmission (English and Sweatt, 1997). TSA (1.65 μM) had no short or long-term effects on NMDA-R—mediated synaptic transmission (n=6, F_{[99,472]}=0.5, p=1). Representative traces immediately before (dashed) and 186 min after (solid) introduction of TSA are shown. Calibration bar indicates 5 msec and 1 mV. In FIG. 8B, slices were exposed to both TSA (1.65 μM) and the NMDA-R antagonist AP5 (50 μM) 20 min prior to LTP induction. After induction of LTP, the AP5 was washed out but slices were still exposed to TSA. Induction of LTP in the presence of TSA was blocked by AP5 (n=8, F_{[89,612]}=0.5, p<1). Representative traces are shown 4 min before (dashed) and 180 min after (solid) LTP induction. Calibration bar indicates 1 mV and 5 msec. In FIG. 8C, depolarization was measured during a 100 Hz tetanus in slices treated with either vehicle (0.05% EtOH, n=10) or TSA (1.65 μM, n=10) for 20 min. No significant difference in area under the curve was seen between slices treated with TSA or vehicle (t=0.6, df=18, p<0.6). Representative depolarizations are shown to the right of the graph. Calibration bar indicates 250 msec and 1 mV. In all panels, error bars are SEM.

FIGS. 9A-9B shows that enhancement of LTP by TSA is dependent on transcription. In FIG. 9A, slices were exposed to either DRB (60 μM, n=12) or DRB+TSA (60 μM+1.65 μM, n=15) beginning 20 min prior to induction of LTP (Arrow) and continuing for 3 h afterward. Slices exposed to DRB show normal early-phase LTP, which decays to basal levels of synaptic efficacy 2 h after high-frequency stimulation. Treatment of slices with DRB (60 μM) completely inhibits the enhancement of LTP normally seen with TSA (1.65 μM). Potentiation of synaptic transmission in the presence of both DRB and TSA decays to unpotentiated levels seen in the DRB-treated slices. In FIG. 9B, slices were exposed to either actinomycin D (25 μM, n=7) or actinomycin D+TSA (25 μM+1.65 μM, n=8) beginning 20 min prior to induction of LTP (Arrow) and continuing for 3 h afterward. Slices exposed to actinomycin D show normal early-phase LTP, which decays to basal levels of synaptic efficacy 2 h after high-frequency stimulation. Treatment of slices with actinomycin D completely inhibits the enhancement of LTP normally seen with TSA. Potentiation of synaptic transmission in the presence of both actinomycin D and TSA decays to unpotentiated levels seen in slices treated with actinomycin-alone. In all panels, representative traces shown are 4 min before (dashed line) and 180 min after (solid line) induction of LTP. Calibration bars in both panels indicate 5 msec and 1 mV.

FIGS. 10A-10C show that sodium butyrate enhanced induction of LTP. The effect of the HDAC inhibitor sodium butyrate was assessed on LTP induced by 2, 100 Hz (1 sec, 20 sec interstimulus interval) tetani. In FIG. 10A, LTP was significantly enhanced (F_{[1,2019]}=7.8, p<0.0001) in slices treated with sodium butyrate (300 μM, n=15) for 40 min (beginning 20 min before LTP induction) relative to slices treated with vehicle (ACSF, n=6). Representative traces 4 min before (dashed line) and 180 min after (solid line) LTP induction are shown next to the graph. Calibration bar indicates 5 msec and 1 mV. In FIG. 10B, there are input/output relationships for slices treated with vehicle or sodium butyrate for 20 min. No significant differences were seen (F_{[2,13]}=3, p<0.1). In FIG. 10C, paired-pulse facilitation was unaffected by treatment of slices with sodium butyrate for 20 min (F_{[1,208]}=0.3, p<0.6). In all panels, error bars are SEM.

FIGS. 11A-11F demonstrate that an inhibitor of HDAC activity enhances formation of long-term memory. Animals were injected with either sodium butyrate (1.2 g/kg) or saline (1.2 mL/kg) 1 h prior to 1-shock contextual fear conditioning. In FIG. 11A, freezing behavior during training was unaffected by injection of sodium butyrate (t=0.4, df=29, p<0.8). In FIG. 11B, animals injected with sodium butyrate displayed significantly more freezing behavior than animals injected with saline when re-exposed to the training chamber 24 h later (t=2, df=29, p<0.05). In FIG. 11C, animals were injected with either sodium butyrate or saline, and then exposed to the training chamber for 3 min. No shock was administered. Spontaneous freezing behavior was unaffected by sodium butyrate (t=1, df=14, p<0.3). In FIG. 11D, animals were re-exposed to the training chamber 24 h after their initial exposure shown in panel C. Sodium butyrate had no affect on spontaneous freezing behavior (t=1.2, df=19, p<0.3). In FIG. 11E, freezing behavior during training was unaffected by injection of sodium butyrate (t=0.6, df=14, p<0.6). F) Animals injected with sodium butyrate displayed similar levels of freezing behavior to animals injected with saline when re-exposed to the training chamber 1 h after training (t=0.2, df=14, p<0.8). Error bars are SEM. Asterisks (*) indicate significant differences as assessed via Student's t-Test.

FIG. 12 provides an exemplary model for role of histone acetylation in long-term memory formation. Formation of long-term memory begins with activation of NMDA-Rs at the plasma membrane. Influx of Ca++ ions activates several different signaling pathways that all converge to activate the MEK/ERK signaling cascade. Upon activation, ERK modulates the activities of several different transcription factors and co-activators. The action of numerous transcription factors and co-activators are integrated by the structure of chromatin and are apparent as an increase in acetylation of histone H3. The changes in chromatin structure ultimately lead to changes in transcription of genes relevant for memory formation. Additionally, latent inhibition training results in changes in chromatin structure apparent as acetylation of histone H4. The modulation of chromatin structure by latent inhibition is not accompanied by ERK activation, and does not lead to acetylation of histone H3. Abbreviations: MNT-R, modulatory neurotransmitter receptor; VGCC, voltage gated Ca++ channel; AC, adenylate cyclase.

FIG. 13 demonstrates that NaB enhances long-term passive avoidance memory. Animals received injections of either saline or NaB 45 min prior to passive avoidance training.

FIG. 14 demonstrates that NaB enhances long-term novel object recognition memory. Animals received injections of either saline or NaB 45 min prior to novel object training.

### DETAILED DESCRIPTION OF THE INVENTION

The term “a” or “an” as used herein the specification may mean one or more. As used herein in the claim(s), when used in conjunction with the word “comprising”, the words “a” or “an” may mean one or more than one. As used herein “another” may mean at least a second or more. Some embodiments of the invention may consist of or consist essentially of one or more elements, method steps, and/or methods of the invention. It is contemplated that any method or composition described herein can be implemented with respect to any other method or composition described herein.

The practice of the present invention will employ, unless otherwise indicated, conventional techniques of molecular biology, microbiology, recombinant DNA, and so forth which are within the skill of the art. Such techniques are explained fully in the literature. See e.g., Sambrook, Fritsch, and Maniatis, MOLECULAR CLONING: A LABORATORY MANUAL, Second Edition (1989), OLIGONUCLEOTIDE SYNTHESIS (M. J. Gait Ed., 1984), ANIMAL CELL CULTURE (R. I. Freshney, Ed., 1987), the series METHODS IN ENZYMOLOGY (Academic Press, Inc.); GENE TRANSFER VECTORS FOR MAMMALIAN CELLS (J. M. Miller and M. P. Calos eds. 1987), HANDBOOK OF EXPERIMENTAL IMMUNOLOGY, (D. M. Weir and C. C. Blackwell, Eds.), CURRENT PROTOCOLS IN MOLECULAR BIOLOGY (F. M. Ausubel, R. Brent, R. E. Kingston, D. D. Moore, J. G. Siedman, J. A. Smith, and K. Struhl, eds., 1987), CURRENT PROTOCOLS IN IMMUNOLOGY (J. E. Coligan, A. M. Kruisbeek, D. H. Margulies, E. M. Shevach and W. Strober, eds., 1991); ANNUAL REVIEW OF IMMUNOLOGY; as well as monographs in journals such as ADVANCES IN IMMUNOLOGY.

I. Definitions

The term “attention deficit/hyperactivity disorder” as used herein refers to a disorder in an individual, which may be an infant, child, or adult, in which: one or more symptoms of inattention have persisted for at least 6 months to a degree that is maladaptive and inconsistent with developmental level; and/or one or more symptoms of hyperactivity-impulsivity have persisted for at least 6 months to a degree that is maladaptive and inconsistent with developmental level. In particular embodiments, some hyperactive-impulsive or inattentive symptoms that caused impairment were present before age 7 years; some impairment from the symptoms is present in two or more settings (e.g. at school, work, or home); there is clear evidence of clinically significant impairment in social, academic, or occupational functioning; the symptoms do not occur exclusively during the course of a Pervasive Developmental Disorder, Schizophrenia, or other Psychotic Disorder, for example, and are not better accounted for by another mental disorder (e.g. Mood Disorder, Anxiety Disorder, Dissociative Disorder, or a Personality Disorder, for example).

Furthermore, although ADD is officially called Attention-Deficit/Hyperactivity Disorder, or AD/HD (American Psychiatric Association, 1994), in some embodiments it may be referred to as ADD or A.D.D. or ADHD. In specific embodiments, AD/HD [A.D.D. or ADHD] is divided into three subtypes, according to the main features associated with the disorder: inattentiveness, impulsivity, and hyperactivity. The three subtypes include the following: AD/HD Predominantly Combined Type; AD/HD Predominantly Inattentive Type; and AD/HD Predominantly Hyperactive-Impulsive Type.

These subtypes take into account that some individuals with AD/HD have little or no trouble sitting still or inhibiting behavior, but may be predominantly inattentive and, as a result, have great difficulty getting and/or staying focused on a task or activity. Others with AD/HD may be able to pay attention to a task but lose focus because they may be predominantly hyperactive-impulsive and, thus, have trouble controlling impulse and activity. The most prevalent subtype is the Combined Type, wherein these individuals exhibit significant symptoms of all three characteristics.

The term “cognition” as used herein refers to the mental process of knowing, including aspects such as awareness, perception, reasoning, and judgment. It may also be referred to as the operation of the mind by which one becomes aware of objects of thought or perception, including all aspects of perceiving, thinking, and remembering.

The term “dementia” as used herein refers to cognitive impairment such that an extent of normal independent function is impossible; for instance, it may refer to the state wherein one can no longer successfully manage their own finances or provide for their own basic needs.

The term “illicit/illegal drug” as used herein refers to a composition that is not sanctioned by custom or law, particularly in the United States. Specific drugs include marijuana, cocaine, heroin, methamphetamine, inhalants, Ecstasy (MDMA, methylenedioxymethamphetamine), Rohypnol (flunitrazepam), GHB (gamma hydroxybutyrate), and ketamine (ketamine hydrochloride), for example.

The term “long-term memory” as used herein refers to memory that is retained over long periods of time, such as more than about two hours.

The term “memory” as used herein refers to that mental ability by which sensations, impressions, and ideas are recalled. In further specific embodiments, the term memory is also referred to herein as forgetfulness or amnesia, and loss of memory may be referred to as impaired memory.

The term “mental retardation” as used herein is characterized by having a significantly below-average score on a test of mental ability or intelligence and/or by having limitations in the ability to function in areas of daily life, such as communication, self-care, and getting along in social situations and school activities. In specific embodiments, the present invention encompasses mental retardation in individuals having cognitive disability. In additional embodiments, the definition of “mental retardation” as used herein approaches the definition of that of the American Association of Mental Retardation, in which mental retardation is a disability characterized by significant limitations both in intellectual functioning and in adaptive behavior as expressed in conceptual, social, and practical adaptive skills.

The term “mild cognitive impairment” as used herein refers to persistent memory problems of a severity to interfere with normal daily routine.

The term “substantially normal memory faculty” as used herein refers to an individual's memory ability being substantially average or better than average in ability compared to the memory ability among a population of individuals. In specific embodiments, an individual having a substantially normal memory faculty is referred to as an individual having a normal Intelligence Quotient (IQ), such as one having an IQ of about 70 or more.

The term “substantially sub-normal memory faculty” as used herein refers to an individual's memory ability that is less than average in ability compared to the memory ability among a population of individuals. In specific embodiments, an individual having a substantially sub-normal memory faculty is referred to as an individual having an IQ of about 70 or less, which is considered to be the range for mental retardation.

II. The Present Invention

The present invention generally relates to the augmentation of cognition, which in particular embodiments is further defined as the augmentation of memory, such as enhancing a baseline memory of an individual, for example. This may be further defined as augmenting memory that is substantially normal or substantially sub-normal, as in substandard. In a specific aspect of the invention, there is treatment of pathogenically-derived memory capacity or defect or there is treatment of non-pathogenically-derived substandard memory capacity or defect. In specific embodiments, there is augmentation of long-term memory.

The present invention generally relates to augmentation of cognition in the context of regulation of chromatin structure. In particular aspects of the invention, modulation of chromatin structure imparts a mechanism for long-term memory formation. In specific embodiments, the present invention is useful for the amelioration of and/or treatment of memory loss, particularly long-term memory loss.

In specific aspects of the invention, formation of long-term memory is associated with changes in chromatin structure. These changes to the chromatin may be of any kind, including structure and/or function, but in particular embodiments they comprise changes associated with histones, including modulation of their acetylation states. For example, the present inventors identified a large change in the acetylation of histone H3 in area CA1 of the hippocampus one hour after contextual fear conditioning. Therefore, changes in hippocampal chromatin structure during the early periods of long-term memory formation are associated with a learning paradigm.

In specific embodiments, the ERK signaling cascade couples the activity of various cytoplasmic signaling pathways with chromatin state. Activation of either the PKC or PKA pathways in the hippocampus led to increases in acetylation of histone H3 that were completely blocked by an inhibitor of MEK, the kinase upstream of ERK. Therefore, ERK plays a key role in transducing relatively short-term cytoplasmic signaling processes into long-term changes that are effected through changes in chromatin structure and altered gene expression. In specific embodiments of the invention, ERK accomplishes this through at least one of the following: direct regulation of transcription factors, such as Elk-1; indirectly modulating the activity of transcriptional co-regulators, histone acetyltransferases of various forms; and/or through recruiting other histone-modifying enzymes to the transcriptional complex, for example histone acetyltransferases, methyltransferases and kinases.

III. Cognitive Applications

The present invention relates to enhancing cognition in an individual. This may entail augmenting memory, perception, reasoning, or judgment, for example. Thus, the present invention encompasses improvement of memory in specific embodiments or may be used for alternative embodiments, such as improving a learning disability, improving attention span, such as with adult or juvenile attention deficit/hyperactivity disorder, or improving the ability to focus mentally, for example.

A. Memory Embodiments

In embodiments wherein the present invention addresses augmentation of memory, this may be for a person with substantially above-normal memory, substantially normal memory, or substantially sub-normal memory. Sub-normal memory may be the result of normal aging-related impairment and/or it may be due to a pathological condition. For example, memory loss (which in specific embodiments includes amnesia) comprises unusual forgetfulness that can be caused by brain damage due to disease or injury, or it can be caused by severe emotional trauma. The cause may determine whether memory loss comes on slowly or suddenly, and whether it is temporary or permanent. Normal aging may result in difficulty learning new material or requiring longer time or even having an inability to recall learned material. In the embodiments wherein dramatic memory loss occurs, a medical condition may be involved. Specific medical conditions that the present invention may encompass include those related to cognitive impairment generally, such as with mental retardation; AD/HD, and, in some embodiments, memory loss specifically, including the following: aging, dementia, Alzheimer's disease, neurodegenerative illness, bipolar disorder, head trauma or injury, hysteria often accompanied by confusion, seizures, general anesthetics such as halothane, isoflurane, and fentanyl, alcoholism, stroke or transient ischemic attack (TIA), transient global amnesia, drugs such as barbiturates or benzodiazepines, electroconvulsive therapy (especially if prolonged), temporal lobe brain surgery, brain masses (caused by tumors or infection), Herpes encephalitis, other brain infections, depression, alcoholism, and/or AIDS-related cognitive impairment, for example.

Specific symptoms of memory loss may include one or more of the following: the inability to recall events from the past (such as with impaired long-term memory); the inability to recall events that occurred prior to a specific experience (anterograde amnesia); the inability to recall events that occurred soon after a specific experience (retrograde amnesia); the tendency to make up stories to cover gaps in memory (confabulation); and/or the presence of low moods that impair concentration.

The loss of memory in an individual treated with compositions and/or methods of the present invention may be minimal or it may be considerable. The memory loss may have been getting worse over years; it may have been developing over weeks or months; it may have been present all the time; or there may have been distinct episodes of amnesia, for example. There also may have been aggravating or triggering factors associated with the memory loss, such as a head injury; an event that was emotionally traumatic; a surgery or procedure requiring a general anesthetic; a pathogenic condition; the use of alcohol by the individual, such as with abuse of alcohol; and/or the use of illegal/illicit drugs, such as with abuse of illegal/illicit drugs, for example.

Currently available yet exemplary diagnostic tests to characterize memory loss may include the following: cerebral angiography; CT scan or MRI of the head; EEG; blood tests (for specific diseases that are suspected); genetic testing; psychometric tests (cognitive tests); or lumbar puncture, for example. In specific embodiments, the invention is employed for an individual suspected of having and/or developing memory loss or an individual that has memory loss.

In particular embodiments, the present invention-encompasses the treatment of mild incognitive impairment (MCI). The term “mild cognitive impairment” as used herein refers to persistent memory problems of a severity to interfere with normal daily routine. When an individual has MCI, they will have more significant memory lapses than would be expected for someone of their age or educational background. However, other mental processes, such as language or attention, are generally unaffected. Typically, the complaints include trouble remembering the names of people they met recently, trouble remembering the flow of a conversation, and an increased tendency to misplace things, or similar problems. For example, an many cases, the individual will be quite aware of these difficulties.

Most importantly, the diagnosis of MCI relies on the fact that the individual is able to perform substantially all of their usual activities successfully, without more assistance from others than they previously needed.

About 10% of people aged 65+ have MCI. About 15% of these will develop dementia each year. Symptoms of MCI may include the following: repeated struggle to remember the names of close colleagues; the missing of appointments more and more frequently; repeating the same information; and/or difficulty to remember details from a recently-viewed event, for example. However, they will be unlikely to experience difficulties with routine activities, such as washing, dressing, shopping and/or maintaining their finances.

B. Attention-Deficit/Hyperactivity Disorder

Another cognitive application for the present invention includes attention-deficit/hyperactivity disorder. Symptoms of ADHD are divided into two main categories: inattention and hyperactivity/impulsivity. A diagnosis of ADHD is based on the number, persistence, and history of ADHD behaviors, and the degree to which they impede an individual's performance in more than one setting. An individual may be exhibiting symptoms of inattention if the individual does the following: ignores details; makes careless mistakes; has trouble sustaining attention in work and/or play; does not seem to listen when directly addressed; does not follow through on instructions; fails to finish; has difficulty organizing tasks and activities; avoids activities that require a sustained mental effort; loses things he or she needs; gets distracted by extraneous noise; and is forgetful in daily activities, for example. An individual may be exhibiting symptoms of hyperactivity associated with ADHD if he or she often does the following: fidgets; has to get up from a seat; runs or climbs when told not to; has difficulty partaking in quiet leisure activities; is in constant activity; and/or talks excessively.

An individual may be exhibiting symptoms of impulsivity if the individual often does the following: answers before questions have been completed; has difficulty waiting his or her turn; and/or interrupts or intrudes on others, for example.

IV. Histone Acetylation Regulators

The present invention encompasses regulators of histone acetylation for enhancement of cognition, including enhancement of memory or treatment of memory loss in an individual. Although any regulator of acetylation is useful in the scope of the invention, in particular embodiments the histone acetylation regulators are histone deacetylase inhibitors or agents that augment histone acetyltransferase activity, for example. In the embodiments wherein histone deacetylase inhibitors are employed to enhance or augment cognition and/or treat memory loss, any suitable histone deacetylase inhibitor may be used.

Nucleosomes, the primary scaffold of chromatin folding, are dynamic macromolecular structures, influencing chromatin solution conformations (Workman and Kingston, 1998). The nucleosome core is made up of histone proteins, H2A, H2B, H3 and H4. Histone acetylation causes nucleosomes and nucleosomal arrangements to behave with altered biophysical properties. The balance between activities of histone acetyl transferases (HAT) and deacetylases (HDAC) determines the level of histone acetylation. Acetylated histones cause relaxation of chromatin and activation of gene transcription, whereas deacetylated chromatin generally is transcriptionally inactive.

More than twelve different HDACs have been cloned from vertebrate organisms. The first three human HDACs identified were HDAC 1 (Taunton et al., 1996), HDAC 2 (Yang et al., 1996) and HDAC 3 (Dangond et al., 1998; Yang et al., 1997; Emiliani et al., 1998) (termed class I human HDACs). Recently class II human HDACs, HDAC 4, HDAC 5, HDAC 6 and HDAC 7 (Kao et al., 2000) have been cloned and identified (Grozinger et al., 1999). All share homology in the catalytic region. A fourth class I human HDAC was recently discovered, and was named HDAC 8, following the order of the appearance of the reports. HDAC9 and HDAC10 were also reported as being class II members. A third class of human histone deacetylases has been described belonging to the Sir2 family of proteins implicated in ageing mechanisms.

A variety of inhibitors for histone deacetylase have been identified. The proposed uses range widely, but primarily focus on cancer therapy (Saunders et al. (1999); Jung et al. (1997); Jung et al. (1999); Vigushin et al. (1999); Kim et al. (1999); Kitazomo et al. (2001); Vigusin et al. (2001); Hoffmann et al. (2001); Kramer et al. (2001); Massa et al. (2001); Komatsu et al. (2001); Han et al. (2001)). They are the subject of an NIH sponsored Phase I clinical trial for solid tumors and non-Hodgkin's lymphoma and also have been shown to increase transcription of transgenes, thus constituting a possible adjunct to gene therapy (Yamano et al. (2000); Su et al. (2000)).

Perhaps the most widely known but exemplary HDAC inhibitor is Trichostatin A, a hydroxamic acid-containing compound. It has been shown to induce hyperacetylation and cause reversion of ras transformed cells to normal morphology (Taunton et al., 1996) and induces immunosuppression in a mouse model (Takahashi et al., 1996). It is commercially available from BIOMOL Research Labs, Inc., Plymouth Meeting, Pa. and from Wako Pure Chemical Industries, Ltd. Also included in the present invention are trichostatins B and C, trapoxins A and B, chlamydocin, sodium butyrate, sodium phenylbutyrate, MS27-275, scriptaid, FR901228, depudecin, oxamflatin, pyroxamide, apicidins B and C, Helminthsporium carbonum toxin, 2-amino-8-oxo-9,10-epoxy-decanoyl, 3-(4-aroyl-1H-pyrrol-2-yl)-N-hydroxy-2-propenamide, suberoylanilide hydroxamic acid, m-carboxycinnamic acid bis-hydroxamide, and FK228. Various HDAC inhibitors are shown in Table 1.

The application is not limited to the listed HDAC inhibitors as, for example, Stemson et al. (2001) identified additional HDAC inhibitors using trichostatin A and trapoxin B as models. Additionally, the following references describe histone deacetylase inhibitors which may be selected for use in the current invention: AU 9,013,101; AU 9,013,201; AU 9,013,401; AU 6,794,700; EP 1,233,958; EP 1,208,086; EP 1,174,438; EP 1,173,562; EP 1,170,008; EP 1,123,111; JP 2001/348340; U.S. 2002/103192; U.S. 2002/65282; U.S. 2002/61860; WO 02/51842; WO 02/50285; WO 02/46144; WO 02/46129; WO 02/30879; WO 02/26703; WO 02/26696; WO 01/70675; WO 01/42437; WO 01/38322; WO 01/18045; WO 01/14581; Furumai et al. (2002); Hinnebusch et al. (2002); Mai et al. (2002); Vigushin et al. (2002); Gottlicher et al. (2001); Jung (2001); Komatsu et al. (2001); Su et al. (2000).

V. Delivery of Histone Acetylation Regulators

The present invention employs histone acetylation regulators for enhancing or improving cognition, such as memory. In specific embodiments, the regulator is a histone deacetylation inhibitor. The compositions of the present invention may be delivered to an individual by any suitable means, and one of skill in the art is aware how to implement such compositions for the methods of the invention.

In particular embodiments, the regulator is delivered in a pharmaceutically acceptable excipient. The regulator may be a polypeptide, peptide, nucleic acid, fatty acid, or small molecule, for example. The regulator may be administered in an aqueous solution or a non-aqueous solution.

Although the regulators may be administered in any suitable form and/or route, in particular they are delivered to an individual orally, by transdermal patch, subdermal injection, intramuscular injection, or by intravenous injection, for example.

VI. Pharmaceutical Compositions and Routes of Administration

Compositions of the present invention will comprise an effective amount of a histone acetylation regulator for therapeutic administration in combination and, in some embodiments, is combined with an effective amount of a compound (second agent) that is therapeutic for the respective appropriate disease or medical condition. Such compositions will generally be dissolved or dispersed in a pharmaceutically acceptable carrier or aqueous medium. The term “effective” as used herein refers to inhibiting an exacerbation in symptoms, preventing onset of a disease, amelioration of at least one symptom, or a combination thereof, and so forth.

The phrases “pharmaceutically or pharmacologically acceptable” refer to molecular entities and compositions that do not produce an adverse, allergic or other untoward reaction when administered to an animal, or human, as appropriate. As used herein, “pharmaceutically acceptable carrier” includes any and all solvents, dispersion media, coatings, antibacterial and antifungal agents, isotonic and absorption delaying agents and the like. The use of such media and agents for pharmaceutical active substances is well known in the art. Except insofar as any conventional media or agent is incompatible with the active ingredients, its use in the therapeutic compositions is contemplated. Supplementary active ingredients, such as other anti-disease agents, can also be incorporated into the compositions.

In addition to the compounds formulated for parenteral administration, such as intravenous or intramuscular injection, other pharmaceutically acceptable forms include, e.g., tablets or other solids for oral administration; time release capsules; and any other form currently used, including cremes, lotions, mouthwashes, inhalants and the like.

The compositions of the present invention may include classic pharmaceutical preparations. Administration of these compositions according to the present invention will be via any common route so long as the target tissue is available via that route. This includes oral, nasal, buccal, rectal, vaginal or topical. Alternatively, administration may be by orthotopic, intradermal, subcutaneous, intramuscular, intraperitoneal or intravenous injection. Such compositions would normally be administered as pharmaceutically acceptable compositions, described supra.

The compositions of the present invention may advantageously be administered in the form of injectable compositions either as liquid solutions or suspensions; solid forms suitable for solution in, or suspension in, liquid prior to injection also may be prepared. These preparations also may be emulsified. A typical composition for such purposes comprises a 50 mg or up to about 100 mg of human serum albumin per milliliter of phosphate buffered saline. Other pharmaceutically acceptable carriers include aqueous solutions, non-toxic excipients, including salts, preservatives, buffers and the like. Examples of non-aqueous solvents are propylene glycol, polyethylene glycol, vegetable oil and injectable organic esters, such as theyloleate. Aqueous carriers include water, alcoholic/aqueous solutions, saline solutions, parenteral vehicles such as sodium chloride, Ringer's dextrose, etc. Intravenous vehicles include fluid and nutrient replenishers. Preservatives include antimicrobial agents, anti-oxidants, chelating agents and inert gases. The pH and exact concentration of the various components in the pharmaceutical are adjusted according to well-known parameters.

Additional formulations are suitable for oral administration. Oral formulations include such typical excipients as, for example, pharmaceutical grades of mannitol, lactose, starch, magnesium stearate, sodium saccharine, cellulose, magnesium carbonate and the like. The compositions take the form of solutions, suspensions, tablets, pills, capsules, sustained release formulations or powders. When the route is topical, the form may be a cream, ointment, salve or spray.

An effective amount of the therapeutic agent is determined based on the intended goal. The term “unit dose” refers to a physically discrete unit suitable for use in a subject, each unit containing a predetermined quantity of the therapeutic composition calculated to produce the desired response in association with its administration, i.e., the appropriate route and treatment regimen. The quantity to be administered, both according to number of treatments and unit dose, depends on the subject to be treated, the state of the subject and the protection desired. Precise amounts of the therapeutic composition also depend on the judgment of the practitioner and are peculiar to each individual.

All of the essential materials and reagents required for therapy may be assembled together in a kit. When the components of the kit are provided in one or more liquid solutions, the liquid solution preferably is an aqueous solution, with a sterile aqueous solution being particularly preferred.

For in vivo use, an agent may be formulated into a single or separate pharmaceutically acceptable syringeable composition. In this case, the container means may itself be an inhalant, syringe, pipette, eye dropper, or other such like apparatus, from which the formulation may be applied to an infected area of the body, such as the lungs, injected into an animal, or even applied to and mixed with the other components of the kit.

The components of the kit may also be provided in dried or lyophilized forms. When reagents or components are provided as a dried form, reconstitution generally is by the addition of a suitable solvent. It is envisioned that the solvent also may be provided in another container means. The kits of the invention may also include an instruction sheet defining administration of the therapeutic composition.

The kits of the present invention also will typically include a means for containing the vials in close confinement for commercial sale such as, e.g., injection or blow-molded plastic containers into which the desired vials are retained. Irrespective of the number or type of containers, the kits of the invention also may comprise, or be packaged with, an instrument for assisting with the injection/administration or placement of the ultimate complex composition within the body of an animal. Such an instrument may be an inhalant, syringe, pipette, forceps, measured spoon, eye dropper or any such medically approved delivery vehicle.

The active compounds of the present invention will often be formulated for parenteral administration, e.g., formulated for injection via the intravenous, intramuscular, subcutaneous, or even intraperitoneal routes. The preparation of an aqueous composition that contains a second agent(s) as active ingredients will be known to those of skill in the art in light of the present disclosure. Typically, such compositions can be prepared as injectables, either as liquid solutions or suspensions; solid forms suitable for using to prepare solutions or suspensions upon the addition of a liquid prior to injection can also be prepared; and the preparations can also be emulsified.

Solutions of the active compounds as free base or pharmacologically acceptable salts can be prepared in water suitably mixed with a surfactant, such as hydroxypropylcellulose. Dispersions can also be prepared in glycerol, liquid polyethylene glycols, and mixtures thereof and in oils. Under ordinary conditions of storage and use, these preparations contain a preservative to prevent the growth of microorganisms.

The pharmaceutical forms suitable for injectable use include sterile aqueous solutions or dispersions; formulations including sesame oil, peanut oil or aqueous propylene glycol; and sterile powders for the extemporaneous preparation of sterile injectable solutions or dispersions. In all cases the form must be sterile and must be fluid to the extent that easy syringability exists. It must be stable under the conditions of manufacture and storage and must be preserved against the contaminating action of microorganisms, such as bacteria and fungi.

The active compounds may be formulated into a composition in a neutral or salt form. Pharmaceutically acceptable salts, include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like.

The carrier can also be a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion and by the use of surfactants. The prevention of the action of microorganisms can be brought about by various antibacterial ad antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

In certain cases, the therapeutic formulations of the invention could also be prepared in forms suitable for topical administration, such as in cremes and lotions. These forms may be used for treating skin-associated diseases, such as various sarcomas.

Upon formulation, solutions will be administered in a manner compatible with the dosage formulation and in such amount as is therapeutically effective. The formulations are easily administered in a variety of dosage forms, such as the type of injectable solutions described above, with even drug release capsules and the like being employable.

For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous and intraperitoneal administration. In this connection, sterile aqueous media which can be employed will be known to those of skill in the art in light of the present disclosure. For example, one dosage could be dissolved in 1 mL of isotonic NaCl solution and either added to 1000 mL of hypodermoclysis fluid or injected at the proposed site of infusion, (see for example, “Remington's Pharmaceutical Sciences” 15th Edition, pages 1035-1038 and 1570-1580). Some variation in dosage will necessarily occur depending on the condition of the subject being treated. The person responsible for administration will, in any event, determine the appropriate dose for the individual subject.

Those of skill in the art will recognize that the best treatment regimens for using a composition of the present invention to provide therapy can be straightforwardly determined. This is not a question of experimentation, but rather one of optimization, which is routinely conducted in the medical arts. For example, in vivo studies in nude mice provide a starting point from which to begin to optimize the dosage and delivery regimes. The frequency of injection will initially be once a wk, as was done some mice studies. However, this frequency might be optimally adjusted from one day to every two weeks to monthly, depending upon the results obtained from the initial clinical trials and the needs of a particular patient. Human dosage amounts can initially be determined by extrapolating from the amount of composition used in mice. In certain embodiments it is envisioned that the dosage may vary from between about 1 mg composition DNA/Kg body weight to about 5000 mg composition DNA/Kg body weight; or from about 5 mg/Kg body weight to about 4000 mg/Kg body weight or from about 10 mg/Kg body weight to about 3000 mg/Kg body weight; or from about 50 mg/Kg body weight to about 2000 mg/Kg body weight; or from about 100 mg/Kg body weight to about 1000 mg/Kg body weight; or from about 150 mg/Kg body weight to about 500 mg/Kg body weight. In other embodiments this dose may be about 1, 5, 10, 25, 50, 75, 100, 150, 200, 250, 300, 350, 400, 450, 500, 550, 600, 650, 700, 750, 800, 850, 900, 950, 1000, 1050, 1100, 1150, 1200, 1250, 1300, 1350, 1400, 1450, 1500, 1600, 1700, 1800, 1900, 2000, 2500, 3000, 3500, 4000, 4500, 5000 mg/Kg body weight. In other embodiments, it is envisaged that higher does may be used, such doses may be in the range of about 5 mg composition/Kg body to about 20 mg composition/Kg body. In other embodiments the doses may be about 8, 10, 12, 14, 16 or 18 mg/Kg body weight. Of course, this dosage amount may be adjusted upward or downward, as is routinely done in such treatment protocols, depending on the results of the initial clinical trials and the needs of a particular patient.

VII. Courses of Therapy

In specific embodiments of the invention, HDAC inhibitors are employed to enhance memory in an individual with substantially normal memory faculty or in an individual with substantially sub-normal memory capacity. When treating an individual with substantially sub-normal memory capacity, the HDAC inhibitor may be utilized at the first sign of clear memory deficiency, such as upon a clear pathogenic state of memory decline. In some embodiments, diagnosed patients that have not received any other therapy are treated, whereas in alternative embodiments another therapy has been applied and the HDAC inhibitor is used as an alternative or additional, such as supplemental, treatment.

In another embodiment, the HDAC inhibitors of the present invention may be used in combination with other agents to improve or enhance the therapeutic effect of either. This process may involve administering both agents to the patient at the same time, either as a single composition or pharmacological formulation that includes both agents, or by administering two distinct compositions or formulations, wherein one composition includes the HDAC inhibitor and the other includes the second agent(s).

The HDAC therapy also may precede or follow the other agent treatment by intervals ranging from minutes to weeks. In embodiments where the other agent and HDAC inhibitor are administered separately, one may prefer that a significant period of time did not expire between the time of each delivery, such that the agent and HDAC inhibitor would still be able to exert an advantageously combined effect. In such instances, it is contemplated that one may administer both modalities within about 12-24 hours of each other and, more preferably, within about 6-12 hours of each other. In some situations, it may be desirable to extend the time period for treatment significantly, however, where several days (2, 3, 4, 5, 6 or 7) to several weeks (1, 2, 3, 4, 5, 6, 7 or 8) lapse between the respective administrations. In other embodiments, it may be desirable to alternate the compositions so that the subject is not tolerized.

Various additional combinations may be employed, HDAC inhibitor therapy is “A” and the secondary agent is “B”:

A/B/A B/A/B B/B/A A/A/B A/B/B B/A/A A/B/B/B
B/A/B/B B/B/B/A B/B/A/B A/A/B/B A/B/A/B A/B/B/A
B/B/A/A B/A/B/A B/A/A/B A/A/A/B B/A/A/A A/B/A/A
A/A/B/A

It is expected that the treatment cycles would be repeated as necessary.

Various drugs for the treatment of memory loss are currently available as well as under study and regulatory consideration. Specific examples are as follows:

Memantine, or Akatinol, is an NMDA receptor agent, which promotes nerve cell viability; Galantamine, or Reminyl, is an agent that raises brain levels of acetylcholine. Some preliminary data suggests that is more effective than the earlier cholinergic agents; Neotropin, as the name suggests, is drug that possibly promotes the growth of nerve cell processes and maintains nerve cell viability; Nootropics, the first class of agents used for treatment of memory loss; Non-steroidal anti-inflammatory agents, or NSAIDS, include drugs such as ibuprofen (Motrin, Advil, etc), as well as the newer cyclo-oxegenase 2 inhibitors (Celebrex and Vioxx); Gingko biloba, a free radical scavenger and possible brain activator; Vaccines that dissolve plaques in the brain are also in development; Certain classes of the B vitamins are felt to be neuroprotective and are being used in clinical trials for treating memory loss; Calcium channel blockers, a class of drugs used to treat illnesses like hypertension and migraine, have been used to treat memory loss; and/or Statins, a class of drugs used to lower cholesterol levels, may reduce amyloid plaque formation and thus may be helpful in some types of memory loss.

VIII. Animal Models

In particular aspects of the invention, animal models are employed for characterization of specific HDAC inhibitors in a clinical scenario. However, in specific aspects animals having no particular genetic or phenotypic background to mimic a condition are used but are instead phenotypically normal; these are subject to behavioral test to characterize memory capacity. Some models may be utilized in more than one context, such as to test for cognitive-associated applications, including mental retardation specifically, for example. Exemplary models are listed below, although any suitable model may be utilized.

A. Model for Cognitive Development

Transgenic mice (TgDyrk1A) overexpressing the full-length cDNA of Dyrk1A comprise a murine model for Down's syndrome (Altafaj et al., 2001). In the Morris water maze, TgDyrk1A mice show a significant impairment in spatial learning and cognitive flexibility, indicative of hippocampal and prefrontal cortex dysfunction. In the more complex repeated reversal learning paradigm, this defect turned out to be specifically related to reference memory, whereas working memory was almost unimpaired.

Zohar et al. (2003) describe a non-invasive closed-head weight-drop mouse model to produce minimal traumatic brain injury (mTBI). The mice were subjected to the Morris water maze in which they suffered profound long-lasting learning and memory deficits that were force- and time-dependent.

B. Model for Memory Loss

Procedures and animal regimens as described herein in Example 2 are exemplary means for a standard contextual fear conditioning state and are utilized to demonstrate that the HDAC inhibitor TSA facilitates enhancement of long-term potentiation.

Bourtchuladze et al. (1994) describe mice with a targeted disruption of the alpha and delta isoforms of CREB being profoundly deficient in long-term memory.

C. Model for ADHD

The neurological mutant whirler mouse, one of several strains of waltzing mice, may be suitable as an animal model for testing studies relative to hyperkinesis (Sackler and Weltman, 1985).

D. Model for Mental Retardation

As reviewed in Kazuki et al. (2003), four approaches have been used to model Down Syndrome: 1) Transgenic mice overexpressing a single gene from Chr 21; 2) YAC/BAC/PAC transgenic mice containing a single gene or genes on Chr 21; 3) Mice with intact/partial trisomy 16, a region with homology to human Chr 21; and 4) Human Chr 21 transchromosomal mice. Kazuki and colleagues also present a new model system for the study of DS using the transchromosomal technology, including the biological effects of an additional Chr 21 in vivo and in vitro.

IX. Screening Methods

In accordance with the present invention, there also are provided methods for screening drugs or drug combinations for efficacy in treating a cognitive-associated condition or enhancing cognition in a substantially normal individual. Primarily, these methods will rely upon the models described above or other suitable models, but they could easily be adapted to any other suitable assay system, both in vitro and in vivo.

In an exemplary assay, an HDAC inhibitor is provided to an experimental animal via an appropriate route. One or more symptoms of a cognitive-associated condition are then assessed and compared to those seen in a similar animal not receiving the inhibitor, e.g., the same animal prior to receiving the inhibitor. Such symptoms include, but are not limited to: dementia symptoms, decreased concentration, memory loss, inability to focus mentally, inability to perform on water maze or T maze tests, impaired contextual fear conditioned responses, impaired: conditioned place preference, alternating T maze, delayed match-to-place, novel object recognition, passive or active avoidance, latent inhibition, memory extinction, or a combination thereof.

A positive result might be interpreted as the diminution of a symptom, the delay, or prevention in appearance of a previously unseen symptom, or the delay or prevention of progression of an existing symptom.

The method may also comprise screening an HDAC inhibitor in combination with another agent. Thus, depending on whether one was more interested in examining the inhibitor, the other agent or the combination, the appropriate control would be an animal untreated with the inhibitor, the other agent, or both, respectively.

The assay may also comprise various other parameters, including timing of administration, varying the dose, and/or assessing toxicity.

### EXAMPLES

The following examples are included to demonstrate preferred embodiments of the invention. It should be appreciated by those of skill in the art that the techniques disclosed in the examples that follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Exemplary Methods and Reagents

Animals. Young adult, male Sprague-Dawley rats (150-200 g) were used for all experiments. Animals were housed under LD 12:12 and allowed access to rodent chow and water ad libitum. Animals were allowed to acclimate to laboratory conditions at least 3 days prior to use in experiments. All procedures were performed with the approval of the Baylor College of Medicine Institutional Animal Care and Use Committee, and according to national guidelines and policies.

Fear Conditioning. Animals were transported to the laboratory at least 30 min prior to fear conditioning. Animals were placed into the training chamber and allowed to explore for 2 min, after which they received an electric shock (1 sec, 0.5 mA). The 2 min-1 sec shock paradigm was repeated for a total of 3 shocks. After the last shock, animals were allowed to explore the context for an additional 1 min prior to removal from the training chamber. For experiments investigating the effect of sodium butyrate on long-term memory, animals were exposed to only 1 shock. Latent inhibition training was performed by placing the animals into the training chamber 2 h prior to administering the standard fear conditioning paradigm outlined above. In experiments where animals received an injection of either saline (1.25 μL/kg) or MK-801 (100 or 300 μg/kg), injections were performed 1 h prior to fear conditioning. In experiments where animals received an injection of either DMSO (2.9 mL/kg) or SL327 (100 mg/kg), injections were performed immediately after the training session. For behavioral experiments, freezing behavior was measured during and either 1 h or 24 h after fear conditioning. Freezing behavior was measured by observing the animals for 2 sec every 10 sec. Age-matched animals that were handled by the experimenter but did not receive any experimental manipulations (naïve) were used as controls in all fear conditioning experiments.

Hippocampus slice preparation. Animals were sacrificed using a rodent guillotine. The brain was immersed in ice-cold cutting saline (CS [in mM]: 110 Sucrose, 60 NaCl, 3 KCl, 1.25 NaH₂PO₄, 28 NaHCO₃, 0.5 CaCl₂, 7 MgCl₂, 5 Glucose, 0.6 Ascorbate) prior to isolation of the caudal portion containing the hippocampus and entorhinal cortex. Transverse slices (400 μm) were prepared with a Vibratome (Vibratome, St. Louis, Mo.). During isolation, slices were stored in ice-cold CS. After isolation, cortical tissue was removed and hippocampal slices were equilibrated in a mixture of 50% CS and 50% artificial cerebrospinal fluid (ACSF [in mM]: 125 NaCl, 2.5 KCl, 1.25 NaH₂PO₄, 25 NaHCO₃, 2 CaCl₂, 1 MgCl₂, 25 glucose) at room temperature (RT). Slices were further equilibrated in 100% ACSF for 45 min at RT, followed by a final incubation in 100% ACSF at 32° C. for 1 h. All solutions were saturated with 95%/5% O₂/CO₂. Slices were treated with the appropriate drugs or vehicle after the last equilibration at 32° C. For experiments investigating the regulation of histone acetylation in vitro, slices from 4 animals were pooled and randomized, and divided into 2 treatment groups: vehicle control and drug treated. For electrophysiology experiments, slices from 1 animal were recorded from at a time. Therefore, each electrophysiology experiment had matched vehicle and drug treated slices from the same animal.

Isolation of Area CA1. For isolation of area CA1 from whole brain, the brain was immersed in oxygenated (95%/5% O₂/CO₂) ice-cold CS immediately after removal from the animal. For isolation of area CA1 from slices of hippocampus, slices were immersed in ice-cold CS immediately after the treatment period. For whole brain and slice dissections, area CA1 was dissected away from other hippocampal subfields under a dissecting microscope; tissue from 2 animals was pooled for each treatment group. Once isolated, Area CA1 was placed in 4 mL ice-cold homogenization buffer ([in mM] 250 sucrose, 50 Tris pH 7.5, 25 KCl, 0.5 PMSF, 1% protease inhibitor cocktail [Sigma, St. Louis, Mo.], 0.9 Na⁺-butyrate) and homogenized for 12 strokes at 4,000 RPM with a Teflon-on-glass grinder (VWR).

Slice electrophysiology. Electrophysiology was performed in an interface chamber (Fine Science Tools, Foster City, Calif.). Oxygenated ACSF (95%/5% O₂/CO₂) was perfused into the recording chamber at a rate of 1 mL/min. Electrophysiological traces were digitized and stored using a Digidata (models 1200 and 1320A) and Clampex software (Axon Instruments, Union City, Calif.). Extracellular stimuli were administered on the border of Area CA3 and CA1 along the Schaffer-collaterals using Teflon coated, bipolar platinum electrodes. fEPSPs were recorded in stratum radiatum with an ACSF-filled glass recording electrode (1-3 MΩ). The relationship between fiber volley and fEPSP slopes over various stimulus intensities was used to assess baseline synaptic transmission. All subsequent experimental stimuli were set to an intensity that evoked a fEPSP that had a slope of 50% of the maximum fEPSP slope. Paired-pulse facilitation was measured at various interstimulus intervals (20, 50, 100, 200, 300 msec). Long-term potentiation (LTP) was induced with 2, 100 Hz tetani (1 sec), with an interval of 20 sec between tetani. Synaptic efficacy was monitored 0.5 h prior to and 3 h following induction of LTP by recording fEPSPs every 20 sec (traces were averaged for every 2 min interval). Drug or vehicle was introduced 20 min prior to LTP induction.

Histone Extraction. All procedures were performed on ice and all solutions were chilled to 4° C. prior to use unless otherwise indicated. All centrifugation steps were performed at 4° C. Tissue homogenates were centrifuged at 7,700×g for 1 min. The supernatant (cytoplasmic fraction) was aspirated and stored at −80° C. The pellet (nuclear fraction) was resuspended in 1 mL 0.4 N H₂SO₄. Histones were acid-extracted from the nuclear fraction for 30 min. Acid extracts were centrifuged at 14,000×g for 10 min. The supernatant was transferred to a fresh tube and proteins were precipitated with the addition of 250 μL 100% trichloroacetic acid containing 4 mg/mL deoxycholic acid (Na⁺-salt, Sigma) for 30 min. Precipitated proteins were collected by centrifugation at 14,000×g for 30 min. The supernatant was discarded and the protein pellet was washed with 1 mL of acidified acetone (0.1% HCl) followed by 1 mL of acetone for 5 min each. Protein precipitates were collected between washes by centrifugation (14,000×g, 5 min). The resulting purified proteins were resuspended in 10 mM Tris (pH 8) and stored at −80° C.

Western Blotting. Loading buffer was added (final concentration: 6.25 mM Tris pH 6.8, 2% SDS, 10% glycerol, 1.25% 2-mercaptoethanol, 0.1% bromophenol blue) and samples were incubated at RT for 20 min prior to SDS-PAGE. Samples were run on a discontinuous polyacrylamide gel consisting of a 20% acrylamide resolving and a 4% acrylamide stacking gel, after which proteins were transferred to PVDF membranes for immunoblotting. Membranes were blocked in 3% BSA in TTBS ([in mM]: 150 NaCl, 20 Tris pH 7.5, 0.05% Tween-20) for 45 min at RT. Membranes were incubated in primary antibodies overnight at 4° C., and in horseradish peroxidase conjugated secondary antibodies for 2.5 h at RT. Immunolabeling of membranes was detected via chemiluminescence (ECL, Amersham, Piscataway, N.J.; SuperSignal, Pierce, Rockford, Ill.). Luminescence was recorded with BioMax MR film (Kodak Scientific Imaging Systems, Rochester, N.Y.), digitized (Epson Perfection 1240U) and integrated densities of each band quantified with ImageJ (NIH, Bethesda, Md.). Several exposures were captured for each immunoblot to ensure that all densitometry was performed on images taken in the linear exposure range.

Antibodies. The primary antibodies used, and their dilutions were as follows: Anti-MAP kinase 1/2 (1:1,000, Upstate), Anti-Phospho-p44/42 MAP kinase (Thr202/Tyr204, 1:1,000, Cell Signaling), Anti-Histone H3 (mouse monoclonal, 1:500, Upstate), Anti-acetyl Histone H3 (Lys14, 1:1,000, Upstate), Anti-Histone H4 (1:500, Upstate), Anti-acetyl Histone H4 (Lys5/Lys8/Lys12/Lys16, 1:1,000, Upstate). The host for all primary antibodies was rabbit unless otherwise specified. The secondary antibodies were horseradish peroxidase conjugated goat Anti-IgG heavy and light chain (Jackson ImmunoResearch, West Grove, Pa.).

Statistical analysis. All western blotting data was analyzed using a Kruskal-Wallis ANOVA followed by Dunn's multiple comparison test. Analysis of freezing behavior between the contextual fear conditioning and latent inhibition training paradigms, and between saline and sodium butyrate injections were performed with a Student's t-Test. The effect of MK-801 on freezing behavior was analyzed with a one-way ANOVA, followed by Newman-Keuls multiple comparison test. Input/output relationships were analyzed using either a single exponential function [Y=Slope_{MAX}×1−e−^{K×X}] or a linear regression where appropriate. Parameters of the equations used to fit input/output relationships were compared using an F test. Paired-pulse facilitation (PPF) and LTP were analyzed via 2-way ANOVA with repeated measures. For analysis of LTP, data acquired before and after tetani were analyzed separately. Post-hoc comparisons after 2-way ANOVA were made using the method of Bonferroni. Significance for all tests was set at p<0.05.

### Example 2

### Regulation of Histone Acetylation In Vivo by Contextual Fear Conditioning

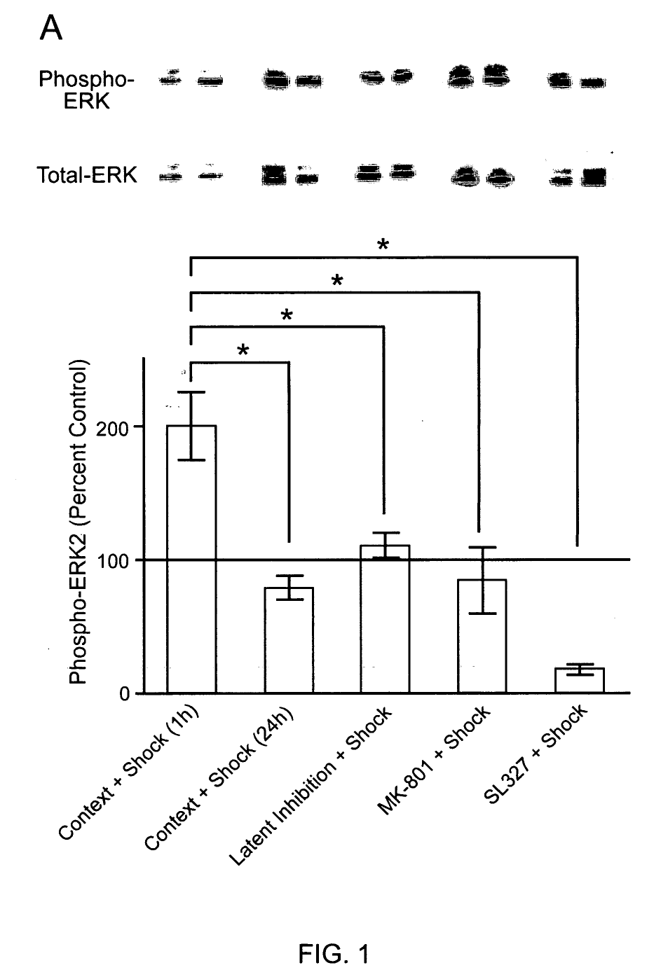

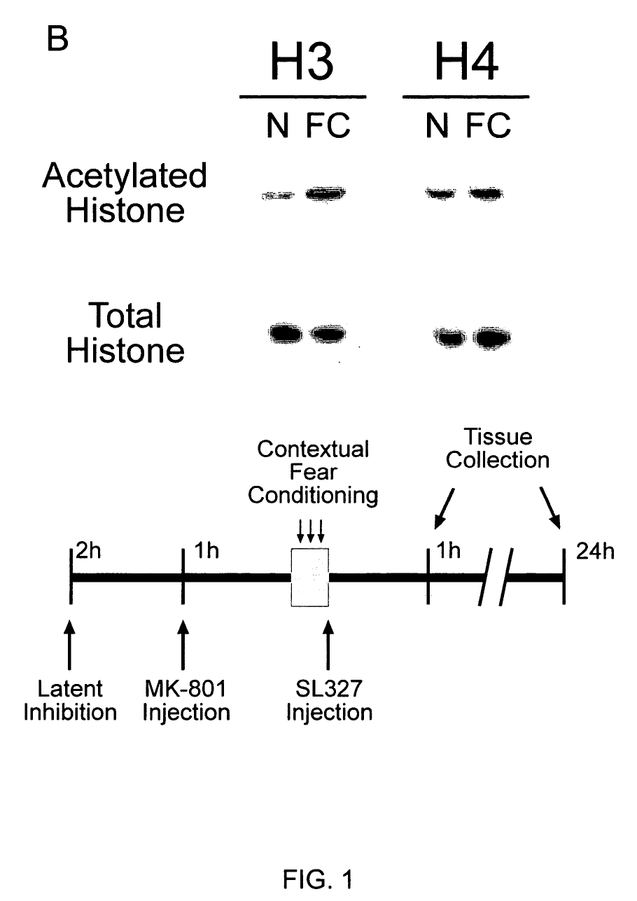

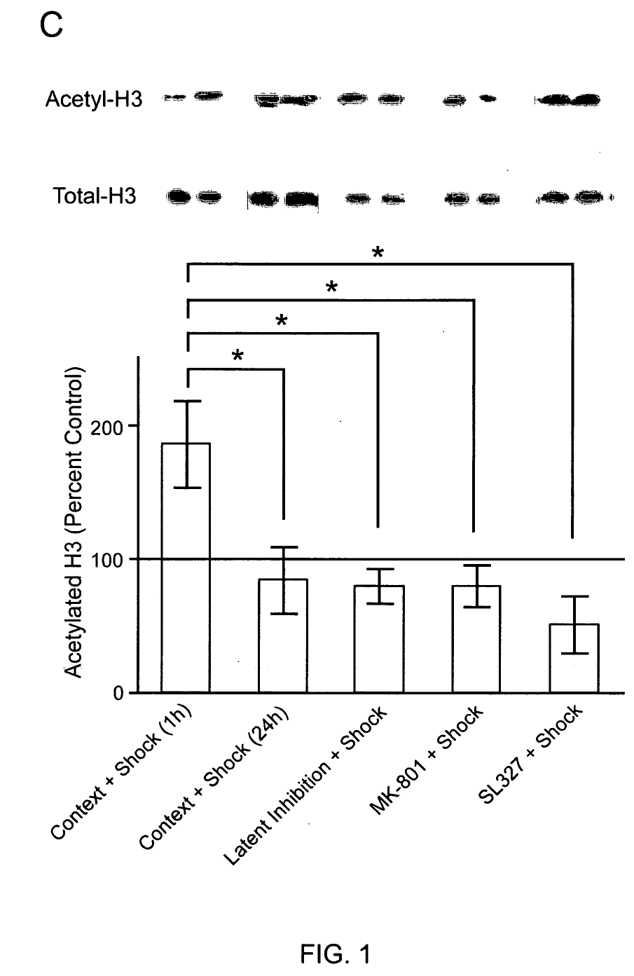

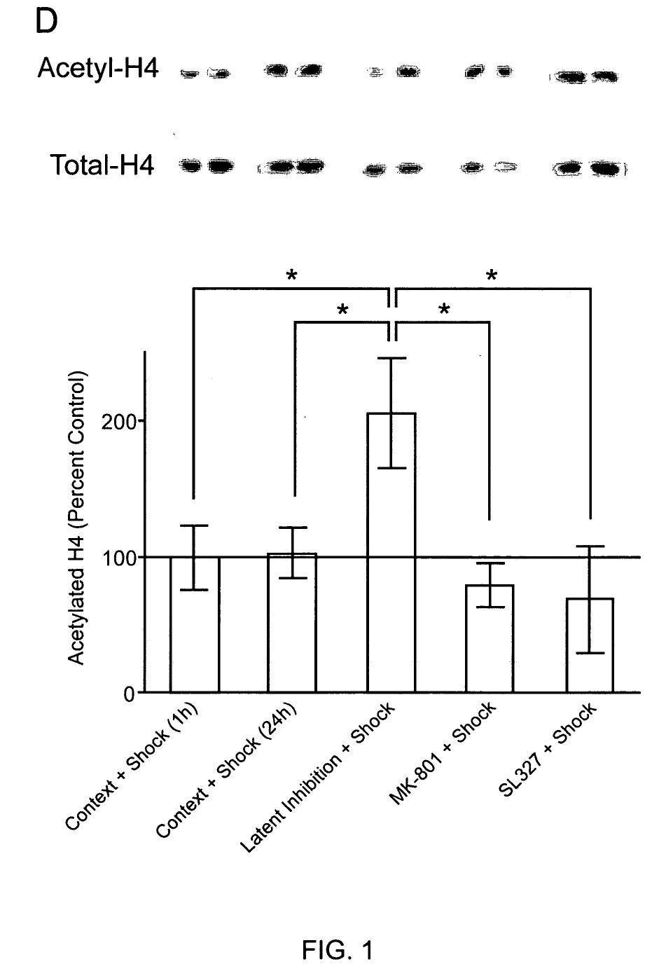

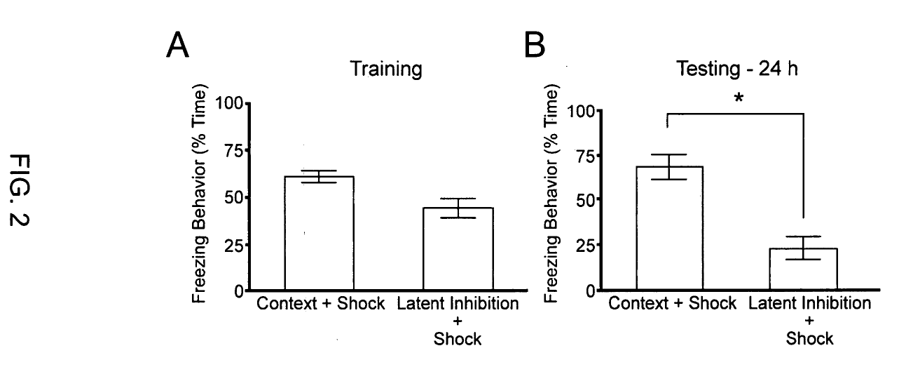

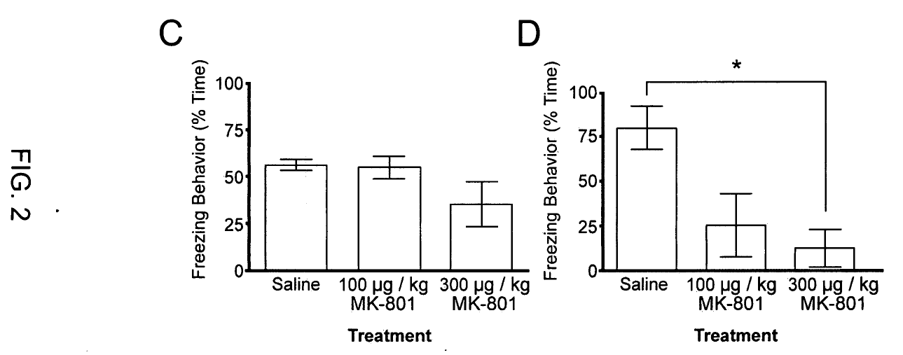

Contextual fear conditioning requires ERK activity and leads to an increase in the activity of ERK2 in area CA1 of the hippocampus of rats 1 h after training (Atkins et al., 1998). The present inventors confirmed that the behavioral methods employed in the current studies also led to increases in ERK2 activity in area CA1 of the hippocampus. Contextual fear conditioning increased levels of phospho-ERK2 (P-ERK2; FIG. 1A) without affecting levels of P-ERK1 (data not shown) when measured 1 h after fear conditioning. The increase in P-ERK2 was not apparent 24 h after training (FIG. 1A), indicating that the regulation of ERK2 was transient. Injection of animals with the NMDA-receptor (NMDA-R) antagonist MK-801 1 h prior to fear conditioning blocked the increase in P-ERK2 normally seen 1 h after fear conditioning (FIG. 1A) and the formation of contextual fear memory (FIG. 2C-D). Moreover, injection of animals with the MEK inhibitor SL327 immediately after fear conditioning, a treatment that prevents formation of long-term contextual fear memory (Atkins et al., 1998), also blocked the increase in P-ERK2 normally seen 1 h after fear conditioning (FIG. 1A). These results indicate that our methods are comparable to previously published reports of biochemical changes associated with fear conditioning in the hippocampus. Furthermore, the results demonstrate that the increase in P-ERK2 seen after fear conditioning requires the activation of NMDA-Rs and the upstream kinase in the ERK cascade, MEK.

The above results indicate that ERK2 phosphorylation was induced by formation of a long-term memory where an animal associates a novel context with a noxious stimulus (footshock). In other embodiments, however, it was possible that the regulation of ERK2 was due to either formation of new spatial memories upon exposure to the novel context, or was a stress response to the footshock alone in the absence of associative memory formation. A latent inhibition training paradigm was used to determine whether either alternative possibilities were contributing to the regulation of ERK2 1 h after fear conditioning.

Latent inhibition occurs when an animal is pre-exposed to a novel context prior to receiving an unconditioned stimulus (electric shock) in that context. Latent inhibition is unique in that the animal does not form an association between the noxious stimulus and the novel context, yet the actual latent inhibition is context specific (Lubow, 1973; Impey et al., 1998) (FIGS. 2A and 2B). Therefore, in latent inhibition training the animal has formed a spatial memory that blocks the formation of an associative contextual fear memory.

Animals were placed into the novel training context 2 h prior to receiving the 3-shock context fear conditioning training paradigm. The latent inhibition training procedure inhibited formation of long-term contextual fear memory (FIGS. 2A and 2B). Exposure of animals to the latent inhibition paradigm blocked the increase in hippocampal P-ERK2 that normally occurs 1 h after fear conditioning (FIG. 1A). Taken together with previously published results (Atkins et al., 1998), these results indicate that the regulation of ERK2 after contextual fear conditioning was specific for the formation of associative contextual fear memories. The lack of regulation of ERK2 after latent inhibition training suggests that regulation of ERK2 in area CA1 of the hippocampus is not involved in the formation of latent inhibition memory, and is consistent with the hypothesis that latent inhibition is independent of ERK2.

Formation of long-term contextual fear memory requires transcription in area CA1 of the hippocampus, and a wide variety of transcripts are regulated with contextual fear conditioning, especially 1 h after conditioning (Levenson et al., 2004). The present inventors investigated whether there might be changes in chromatin structure 1 h after fear conditioning, which is a critical period when transcription occurs during long-term memory formation in the hippocampus (Igaz et al., 2002). The present inventors observed significant increases in the acetylation of histone H3 on Lys14 in Area CA1 of the hippocampus 1 h after contextual fear conditioning (FIGS. 1B and 1C). The increase in H3 acetylation was not apparent 24 h after conditioning (FIG. 1C), indicating that the regulation of H3 was transient, and restricted to periods where critical phases of memory-associated transcription occur (Igaz et al., 2002). Similar to behavioral fear conditioning, the increase in acetylation of histone H3 required the activation of NMDA-Rs and MEK, as injection of animals with either MK-801 or SL327 inhibited the increase in H3 acetylation (FIG. 1C). In addition, the increase in acetylation of histone H3 was specific for the formation of associative contextual fear memory; latent inhibition training blocked the increase in acetylation of histone H3 normally seen with fear conditioning (FIG. 1B, p<0.05). These results indicate that the increase in acetylation of histone H3 on Lys14, 1 h after fear conditioning, is specific for formation of NMDA-R- and ERK—dependent associative contextual fear memory.

To assess whether regulation of histone acetylation was specific to histone H3, or might be a general phenomenon, the acetylation of histone H4 was also investigated. The antibody we used for these experiments detects any combination of Lys acetylation among the 4 different residues in the N-terminus of histone H4. Acetylation of histone H4 was not significantly affected by contextual fear conditioning when measured either 1 h or 24 h after, or by blockade of NMDA-Rs or MEK (FIGS. 1B and 1D). Interestingly, latent inhibition training resulted in significant increases in acetylation of histone H4 (FIG. 1D), suggesting the intriguing possibility that the molecular processes involved in the storage of hippocampus-dependent latent inhibition memory occur through selective regulation of transcription associated with histone H4. Overall, these results demonstrate differential regulation of hippocampal histone acetylation depending on the specific type of memory formed.

### Example 3

### Regulation of Histone Acetylation In Vitro by NMDA-RS

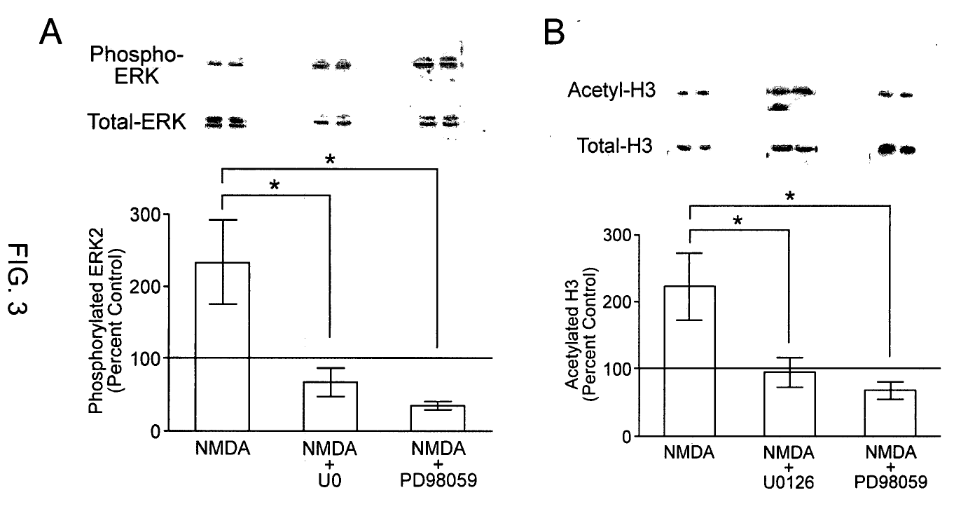

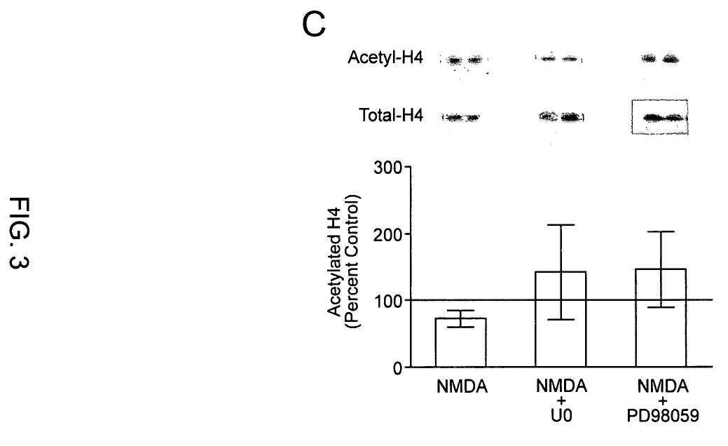

ERK is known to be involved in hippocampus-dependent memory formation, including contextual fear conditioning (Atkins et al., 1998; Selcher et al., 1999). Moreover, the results indicate that the ERK signaling cascade may be important in the regulation of chromatin structure. Therefore, the present inventors tested whether activation of ERK, like behavioral conditioning itself, might trigger regulation of histone acetylation in area CA1. To ensure that the slice cultures were closely modeling the biochemical changes that occur in vivo, the present inventors first investigated whether ERK and/or histone acetylation was regulated by activation of NMDA-Rs. Treatment of hippocampal slices with NMDA for 10 min induced significant increases in P-ERK2 (FIG. 3A). The increases in P-ERK2 were blocked when slices were pretreated with either U0126 or PD98059 (FIG. 3A), which are inhibitors of MEK. Moreover, treatment of slices with NMDA increased acetylation of histone H3, which was blocked by pretreatment with either U0126 or PD98059 (FIG. 3B). No change in the acetylation of histone H4 was observed by any of the above treatments (FIG. 3C). These results indicate that in area CA1 of the hippocampus, activation of NMDA-Rs leads to increases in P-ERK2 and acetylation of histone H3, and that these events require the upstream kinase in the ERK cascade, MEK.

### Example 4

### Regulation of Histone Acetylation In Vitro by ERK Activation

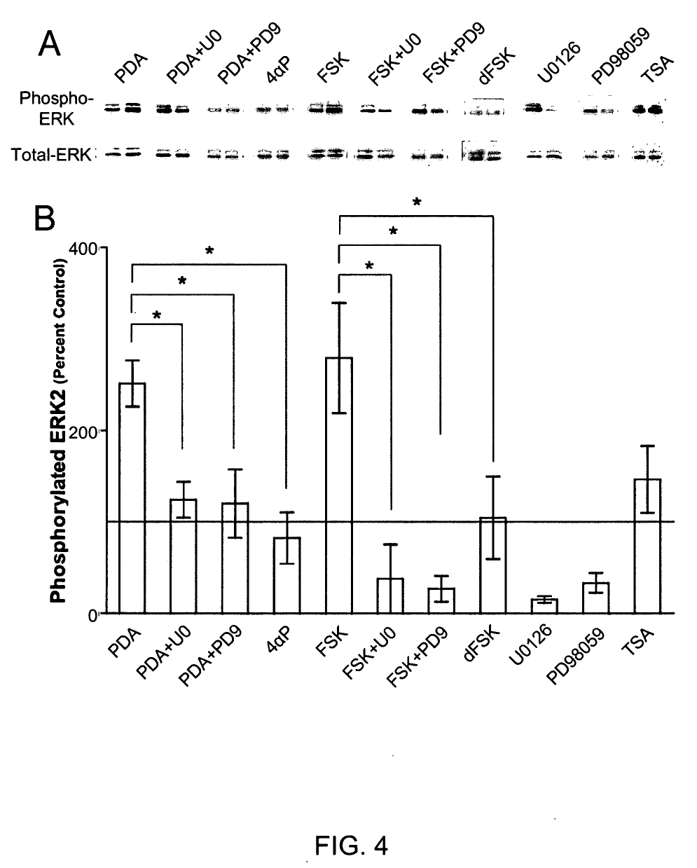

It was next investigated whether activation of either the protein kinase C (PKC) or protein kinase A (PKA) pathways in vitro led to activation of ERK, to determine whether we could use these pathways to study ERK-dependent changes in histone acetylation. Activation of PKC with phorbol 12,13-diacetate (PDA) significantly increased levels of P-ERK1 and P-ERK2 in area CA1 (FIG. 4A-B; P-ERK1 data not shown). Similarly, activation of PKA with forskolin (FSK) significantly increased levels of P-ERK2 (FIG. 4A-B), but not P-ERK1 (data not shown). The effects of both PDA and FSK were specific to the active properties of the drugs, as the respective inactive analogs 4-α-phorbol 12,13-didecanoate (4 αP) and 1,9 dideoxyforskolin (dFSK) had no affect on levels of P-ERK1 or P-ERK2 (FIGS. 4A-4B and data not shown). The effect of both PDA and FSK on P-ERK were blocked by the MEK inhibitors U0126 and PD98059 (FIGS. 4A-4B), confirming that effects of PDA and FSK on ERK were through the activation of the upstream kinase in the ERK cascade, MEK.

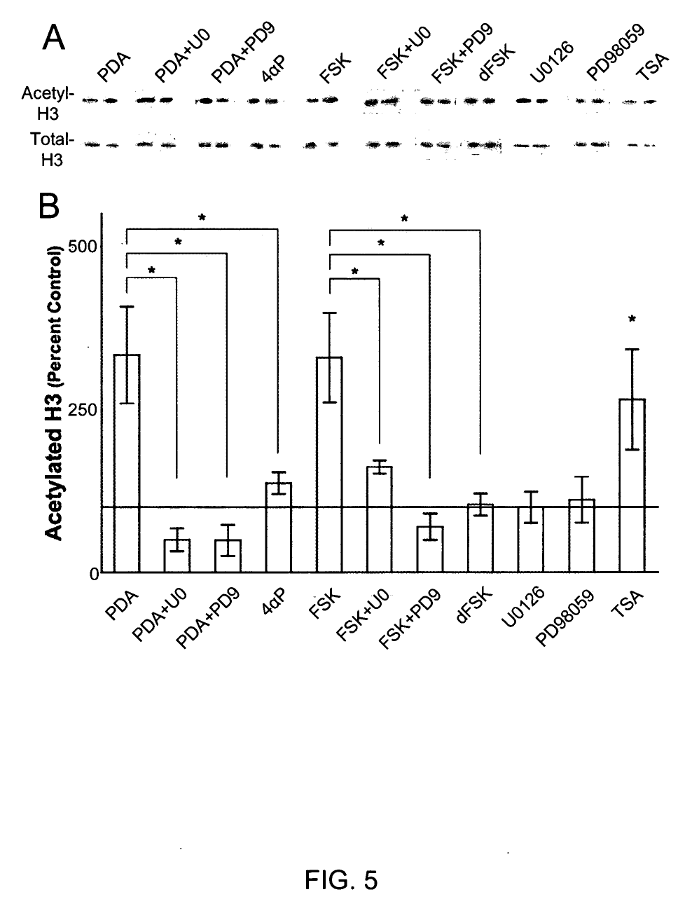

It was next investigated whether activation of ERK via PKC or PKA signaling led to regulation of histone acetylation in hippocampal area CA1. Treatment of slices with either PDA or FSK significantly increased acetylation of histone H3 (FIGS. 5A and 5B). The increases in acetylation of histone H3 were specific for the active properties of the drugs used, as treatment with either 4 αP or dFSK had no affect on the acetylation of histone H3 (FIGS. 5A and 5B). Moreover, pretreatment of slices with the MEK inhibitors U0126 or PD98059 blocked the ability of either PDA or FSK to increase histone H3 acetylation, further confirming that the effects of these drugs were due to their actions on the ERK signaling cascade (FIGS. 5A and 5B). As a positive control, H3 acetylation was measured after exposure of slices to the histone deacetylase inhibitor trichostatin A (TSA). TSA significantly increased acetylation of H3 (FIGS. 5A and 5B) without affecting levels of P-ERK2 (FIGS. 4A and 4B). Thus, activation of ERK in hippocampal area CA1, either through the PKC or PKA signaling pathways, led to increased acetylation of histone H3 in area CA1 of the hippocampus.

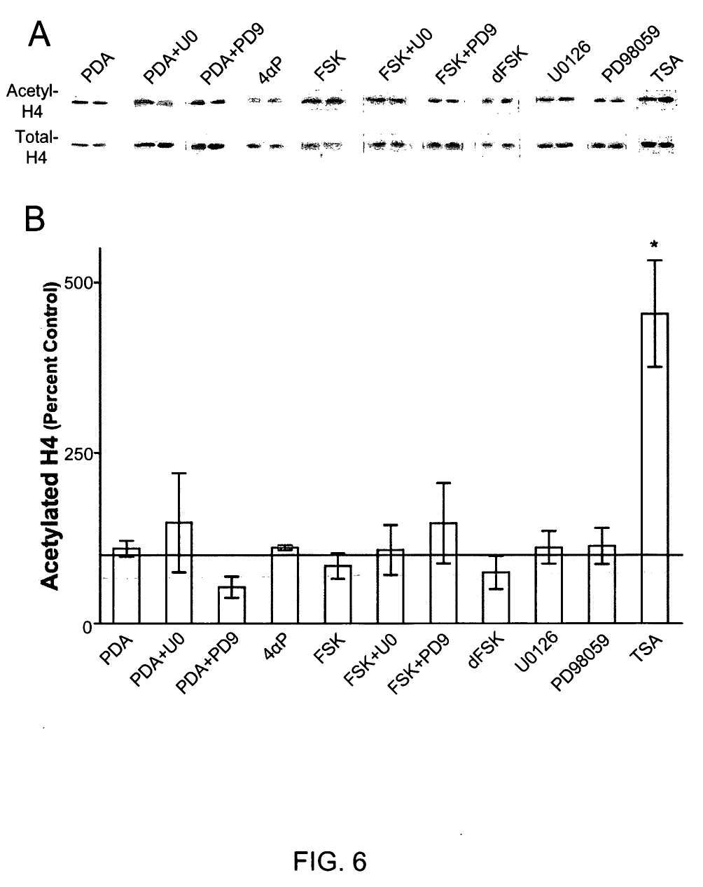

To assess whether activation of ERK in vitro led to a general increase in histone acetylation, levels of histone H4 acetylation were also measured. Activation of either PKC or PKA did not significantly affect the acetylation of histone H4 (FIG. 6B), suggesting that activation of ERK does not regulate acetylation of histone H4. Incubation of slices in the presence of TSA increased the acetylation of histone H4 (FIGS. 6A and 6B), indicating that the antiserum used to measure acetylation of histone H4 was capable of detecting changes in acetylation state. These results support the hypothesis that the overall acetylation state of histone H4 in area CA1 of the hippocampus does not appear to be affected by activation of the ERK signaling cascade.

### Example 5

### Increasing Basal Histone Acetylation Enhances Induction of LTP

The results indicated that acetylation of histone H3 was increased in area CA1 during formation of long-term memory in vivo and with activation of ERK in vitro. This indicated that ERK acts to couple the activity of intracellular signaling cascades to the acetylation state of histones in the nucleus as a mechanism for regulating transcription to induce long-term changes in neuronal function. Induction of long-lasting LTP at Schaffer-collateral synapses in area CA1 of the hippocampus, a candidate mechanism that may contribute to long-term memory formation in vivo, requires activation of ERK in addition to translation and transcription (Stanton and Sarvey, 1984; Frey et al., 1988; Frey et al., 1989; Nguyen et al., 1994; English and Sweatt, 1997; Frey et al., 1996). Therefore, the present inventors sought to determine if elevating levels of histone acetylation facilitated induction of LTP through augmentation of gene transcription by investigating the effect of the histone deacetylase inhibitor TSA on induction of Schaffer-collateral LTP in vitro.

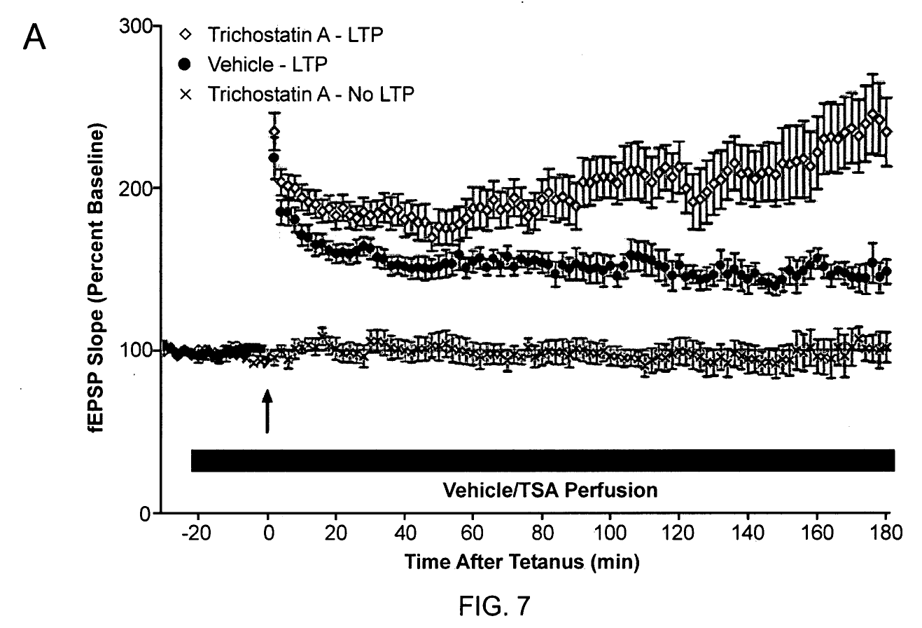

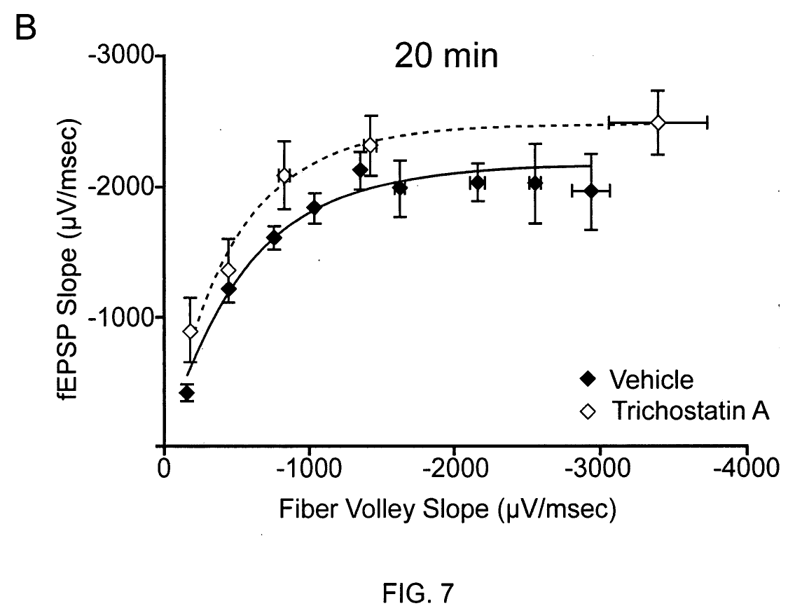

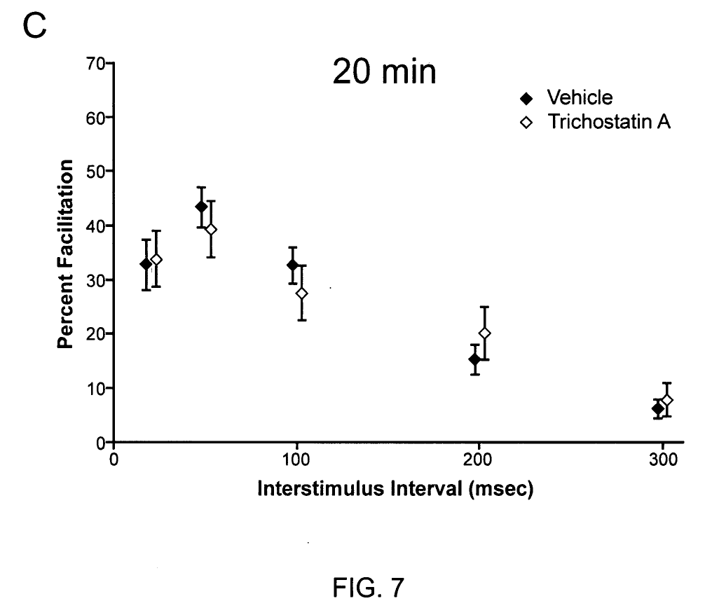

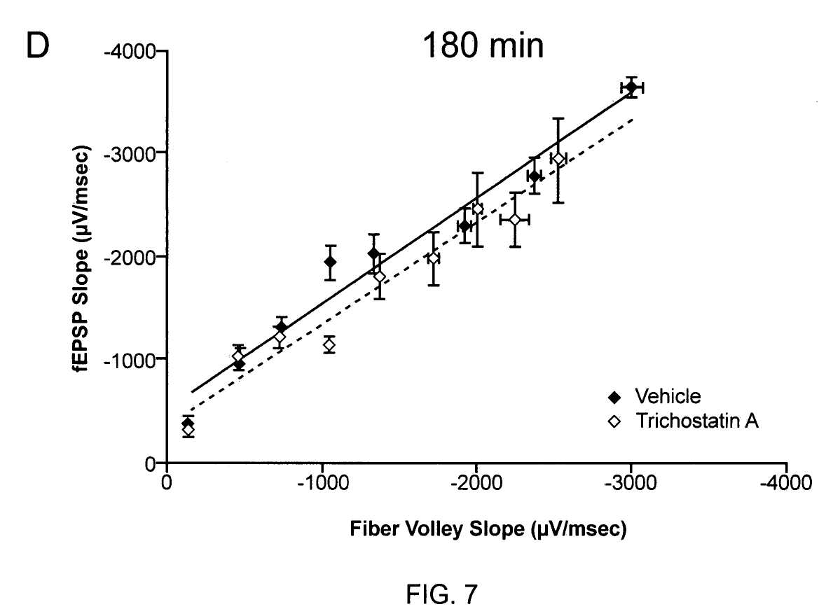

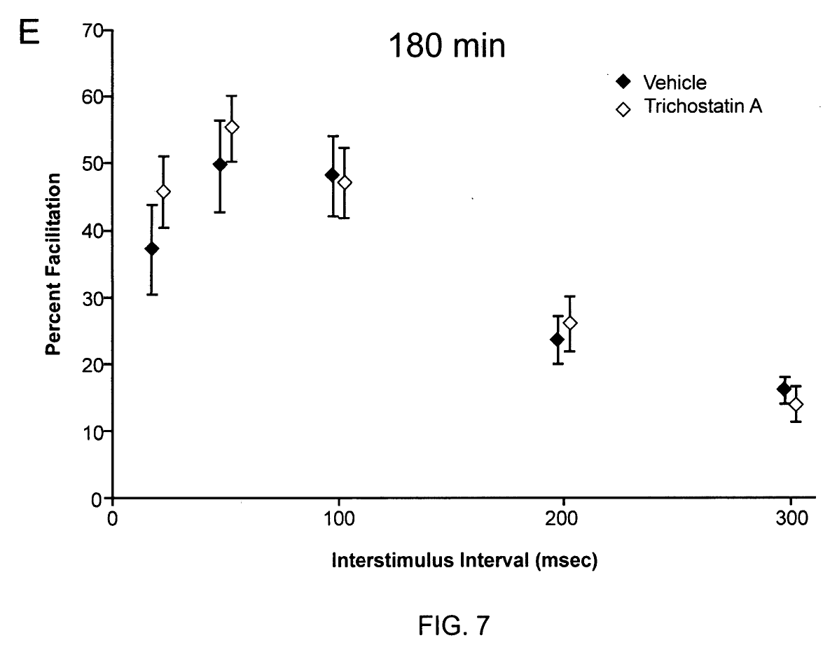

Acute hippocampal slices were maintained in an interface chamber into which either TSA or vehicle was perfused. Extracellular field excitatory postsynaptic potentials (fEPSPs) were recorded in stratum radiatum in area CA1 for 10 min prior to perfusion with drug or vehicle. After the start of perfusion, fEPSPs were measured for another 20 min to ensure that the treatments had no acute effect on baseline synaptic efficacy. After the initial perfusion with either TSA or vehicle, slices were exposed to 2, 100 Hz tetani (1 sec) which resulted in the induction of LTP that lasted at least 3 h (FIG. 7A). Slices treated with vehicle displayed normal LTP while slices treated with TSA exhibited an enhancement of LTP (FIG. 7A). The TSA-mediated enhancement of LTP occurred during both the early (0-120 min) and late (>120 min) phases of LTP (FIG. 7A). These results indicate that an inhibitor of histone deacetylase, which significantly increased acetylation of histones H3 and H4 (FIGS. 5 and 6), can enhance the amount of potentiation triggered by high-frequency stimulation of Schaffer-collateral synapses in area CA1 of the hippocampus.

No studies of the short- or long-term effects of TSA on synaptic properties exist. Therefore, in one embodiment the enhancement of LTP by TSA was due to an indirect effect on synaptic properties, and not through modulation of histone acetylation. To determine whether TSA was indirectly affecting synaptic transmission, the present inventors performed a number of control studies. In the first study, slices were exposed to TSA, and synaptic transmission was monitored for the same time period as in the LTP experiments. TSA had no effect on synaptic transmission in the absence of high-frequency stimulation (FIG. 7A), indicating that TSA was not affecting basal levels of synaptic efficacy or allowing potentiation due to synaptic stimulation at baseline testing frequencies.

To further explore potential effects of TSA on synaptic transmission, slices were exposed to TSA for either 20 min or 3 h, and input/output relationships and paired-pulse facilitation (PPF) were measured. Treating slices for either 20 min or 3 h with TSA had no significant effect on input/output relationships (FIG. 7B; FIG. 7D) or PPF (FIG. 7C; FIG. 7E) relative to control slices treated with the vehicle, suggesting that synaptic connectivity and transmitter release were unaffected by TSA. Taken together, the physiologic results presented thus far indicate that the enhancement of LTP by TSA was not due to indirect effects on basal synaptic transmission.

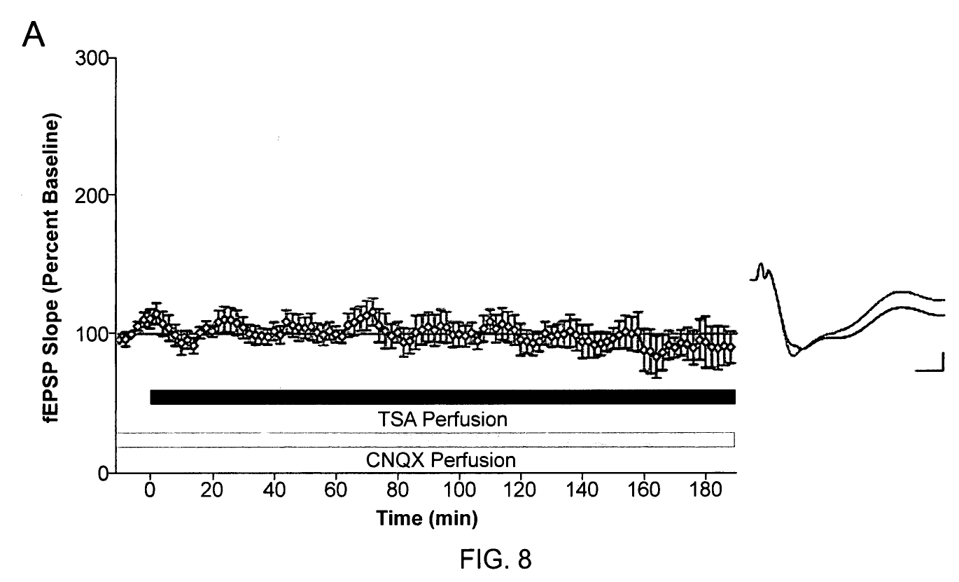

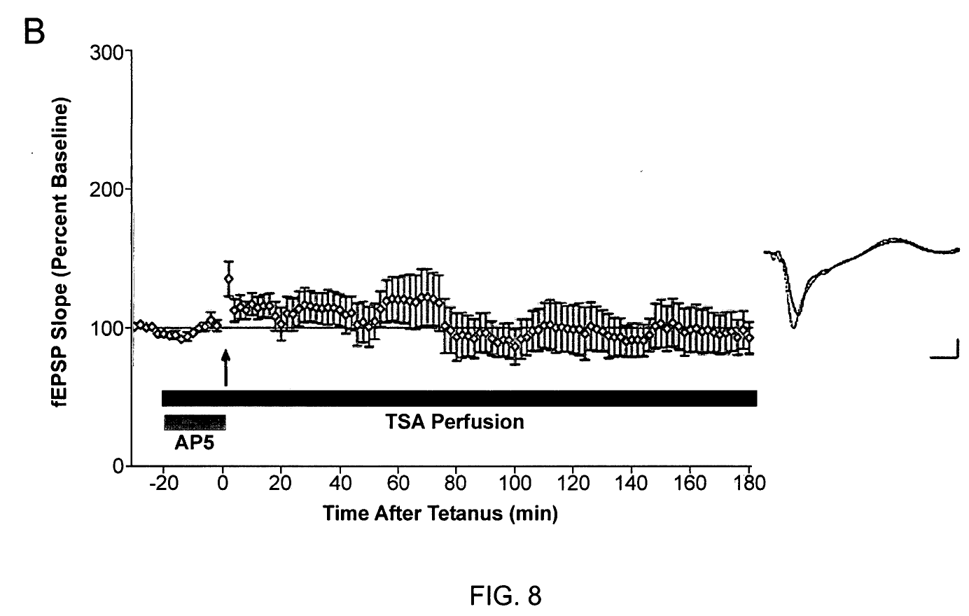

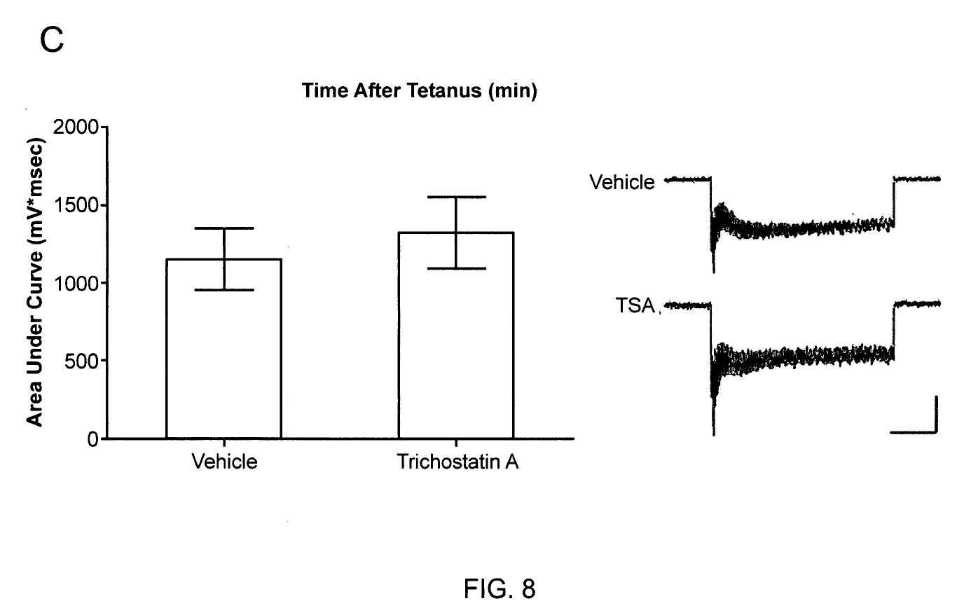

In another series of studies, the present inventors investigated the possibility that TSA was affecting properties of NMDA-Rs. To directly measure any long-term affects of TSA on NMDA-Rs, field potentials were recorded in the presence of the AMPA-R antagonist CNQX to isolate the NMDA-R-dependent component of synaptic transmission. NMDA-R-mediated synaptic transmission was unaffected by chronic exposure to TSA for over 3 h (FIG. 8A). Furthermore, LTP induced in the presence of TSA required NMDA-R activation, as the NMDA-R antagonist AP5 blocked induction of LTP in the presence of TSA (FIG. 8B). To further explore the potential affects of TSA on synaptic transmission, we measured the depolarization that occurs during the 100 Hz tetanus used to induce LTP. No significant difference was observed in the area under the curve of depolarizations induced in the presence or absence of TSA (FIG. 8C). Taken together, the data indicate that TSA has no detectable affect on many different aspects of synaptic transmission that are relevant in the induction of LTP. Therefore, the enhancement of LTP by TSA must occur through an extrasynaptic mechanism.

### Example 6

### The Enhancement of LTP by TSA is Dependent on Transcription

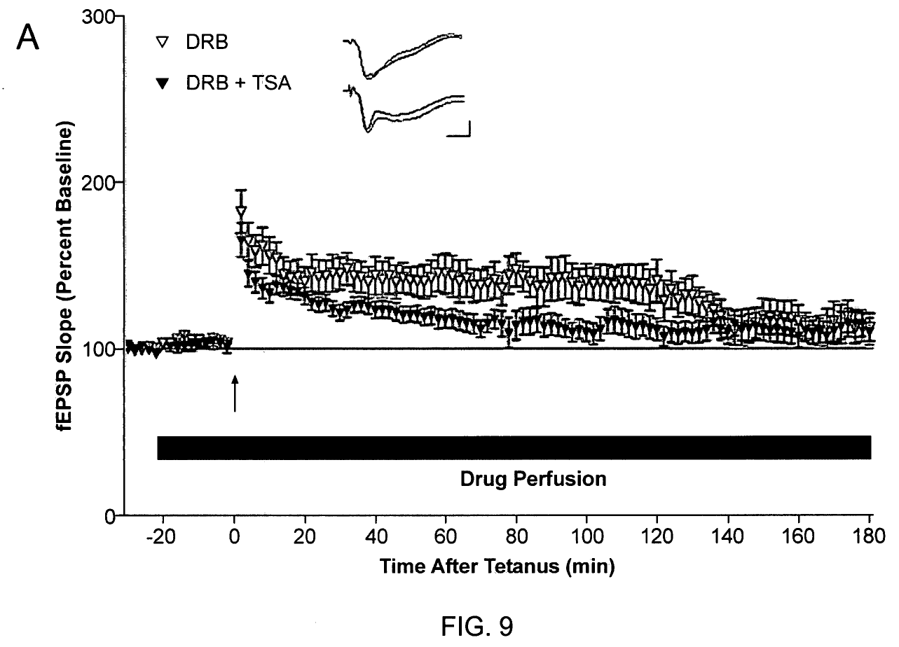

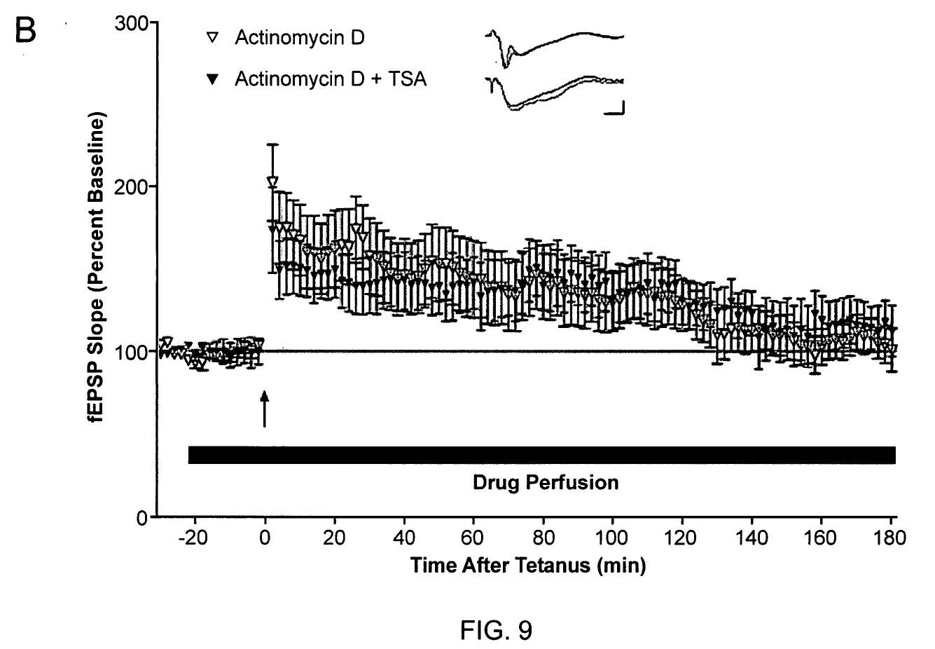

Previous studies investigating the mechanisms underlying LTP in acute slices of hippocampus suggested that transcription is not necessary for potentiation occurring prior to 2 h (Nguyen et al., 1994). However, we observed an effect of TSA immediately after induction of LTP (FIG. 7A). The present inventors therefore sought to determine if the effects of TSA on activity-dependent synaptic potentiation immediately after tetanus were in fact dependent on altered transcription. To this end, the effect of a reversible transcription inhibitor, 5,6-dichlorobenzimidazole riboside (DRB) was tested on the induction of LTP in the presence and absence of TSA. Treatment of slices with DRB had no effect on the expression of LTP up to 2 h after induction (FIG. 9A). However, after 2 h, slices treated with DRB exhibited a decremental LTP that decayed to unpotentiated levels by 3 h after induction (FIG. 9A). Therefore, the LTP induction paradigm used in our study induces a form of LTP whose latter phases are dependent upon transcription for either induction or expression.

Having verified that LTP induced by the procedures is dependent upon transcription, it was next determined if the effects of TSA were also dependent upon transcription. Slices were perfused with both TSA and DRB 20 min prior to the delivery of high-frequency stimulation. When both drugs were present prior to LTP induction, the augmenting effects of TSA on synaptic potentiation were completely blocked at all times (FIG. 9A). Previous studies have indicated that DRB has no effect on basal synaptic transmission (Nguyen et al., 1994). Moreover, detailed analysis of input/output relationships and PPF at 20 min and 3 h after application revealed no significant differences in basal synaptic transmission between slices treated with DRB and slices treated with DRB and TSA (data not shown). Therefore, these results indicate that both the early and late enhancement of LTP by TSA were dependent upon transcriptional modulation.

To further verify that the effect of TSA on LTP was transcription dependent, the effect of another inhibitor of transcription, Actinomycin D (ActD), was tested on the TSA-induced enhancement of LTP. Previous studies have thoroughly documented that ActD has no effect on synaptic transmission (Nguyen et al., 1994). Treatment of slices with ActD for 20 min prior and continuing for 3 h after induction of LTP had a qualitatively similar effect on LTP as compared to DRB (FIG. 9B). In the presence of ActD, LTP appeared normal for the first 2 h after induction, but decayed to basal levels after 2 h (FIG. 9B). Moreover, ActD blocked the effect of TSA on LTP at all times (FIG. 9B), further indicating that the effect of TSA on LTP was due to an effect on transcription. Together with the data obtained using DRB, these results indicate that application of TSA rapidly induces (i.e. within 20 min) the expression of a gene(s) that facilitates either the induction or expression of LTP, but that has no effect on baseline synaptic transmission. In an alternative embodiment, TSA acts to prime the normal transcriptional response to high-frequency synaptic activity, which facilitates the induction or expression of LTP. Overall the studies demonstrate that an agent that modifies histone acetylation, TSA, selectively augments activity-dependent synaptic potentiation via a transcription-dependent mechanism.

### Example 7

### Enhancement of LTP by a Second Histone Deacetylase Inhibitor

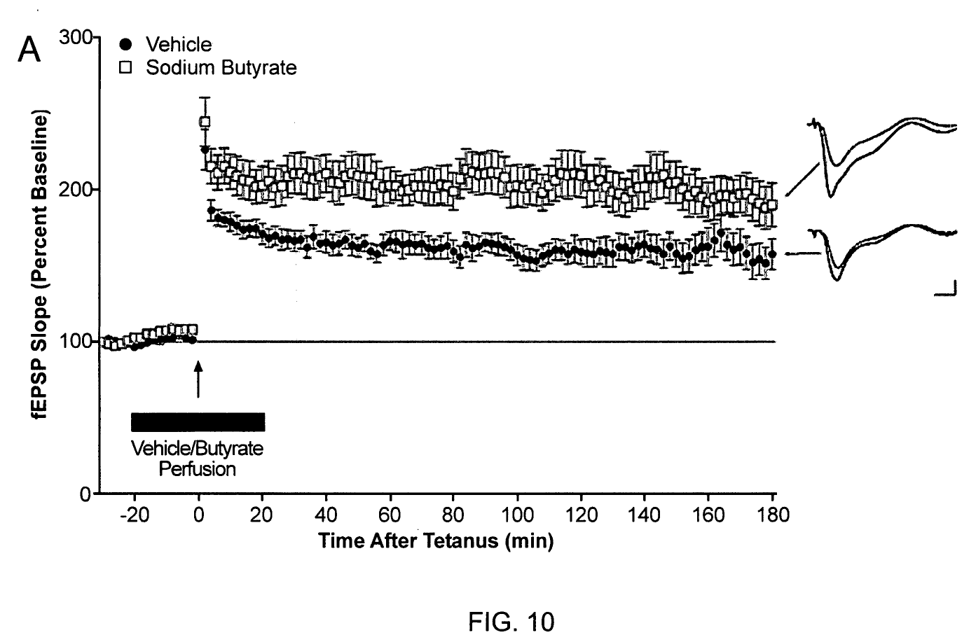

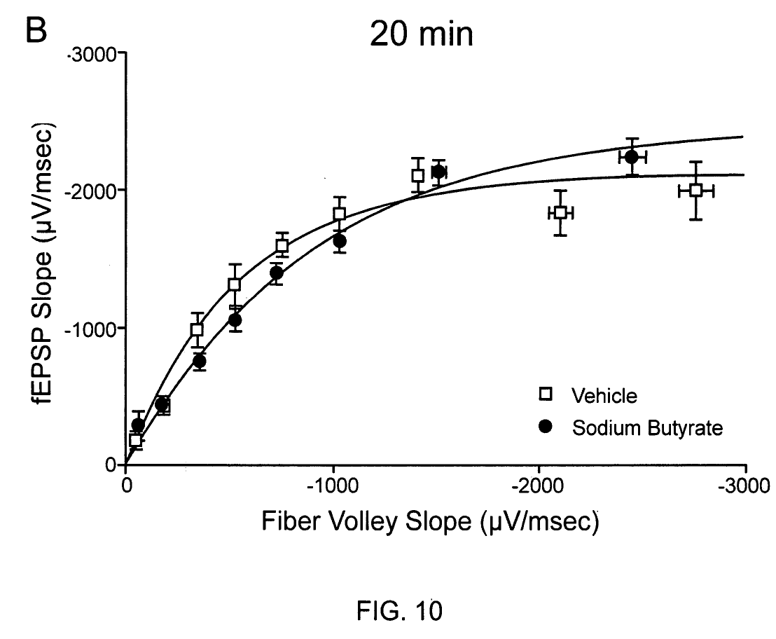

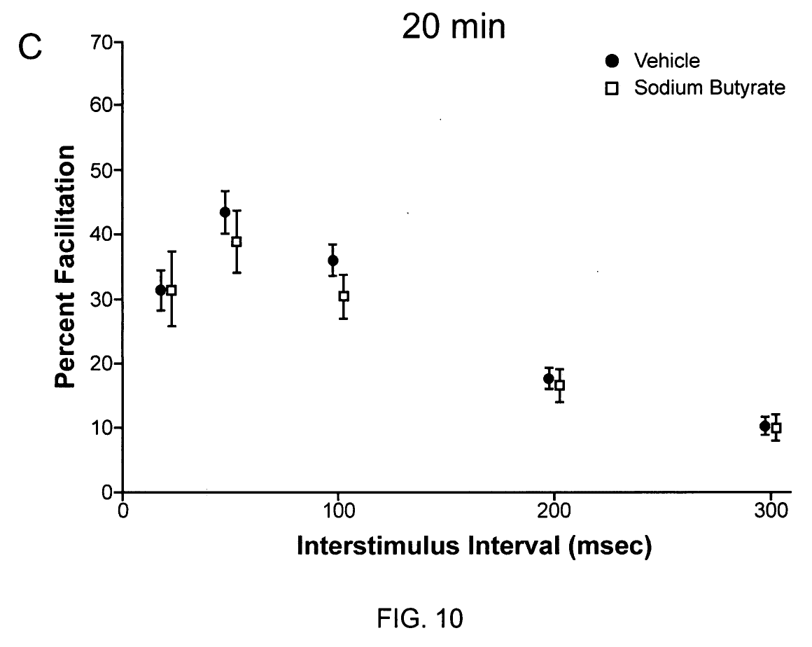

To determine whether the enhancement of LTP by TSA was due to the inhibition of histone deacetylases, we used a structurally dissimilar inhibitor of histone deacetylases, sodium butyrate (NaB). Exposure of slices to NaB for 40 min elevated acetylation of histones H3 and H4 (data not shown), and enhanced induction of LTP (FIG. 10A). The NaB-dependent enhancement of LTP was apparent at all times assayed (FIG. 10A), similar to what was observed for TSA (FIG. 7A). In addition, NaB had no significant affect on input/output relationships (FIG. 10B) or PPF (FIG. 10C), indicating that at the concentration used, NaB did not affect synaptic transmission. These results further indicate that inhibition of histone deacetylases can enhance the induction of LTP.

### Example 8

### An Inhibitor of Histone Deacetylase Enhances Formation of Long-Term Memory

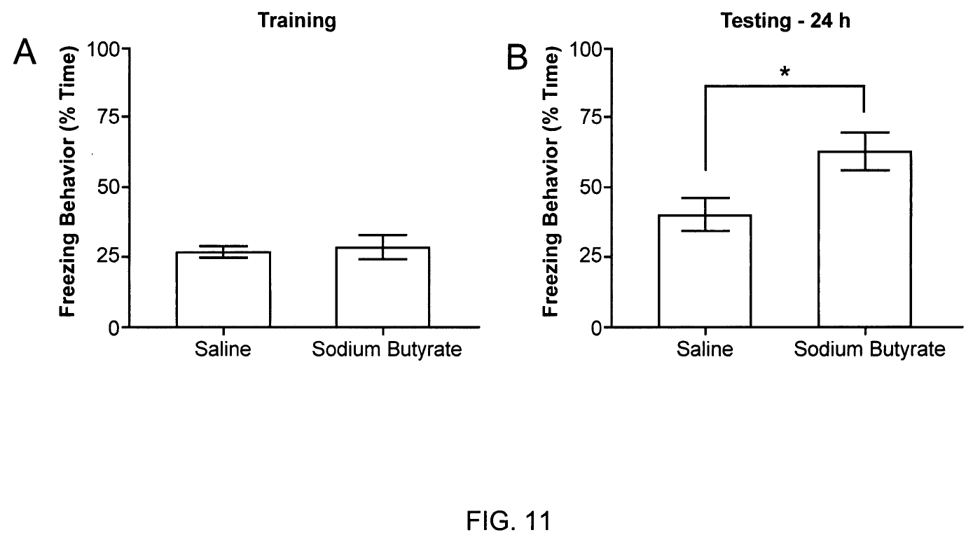

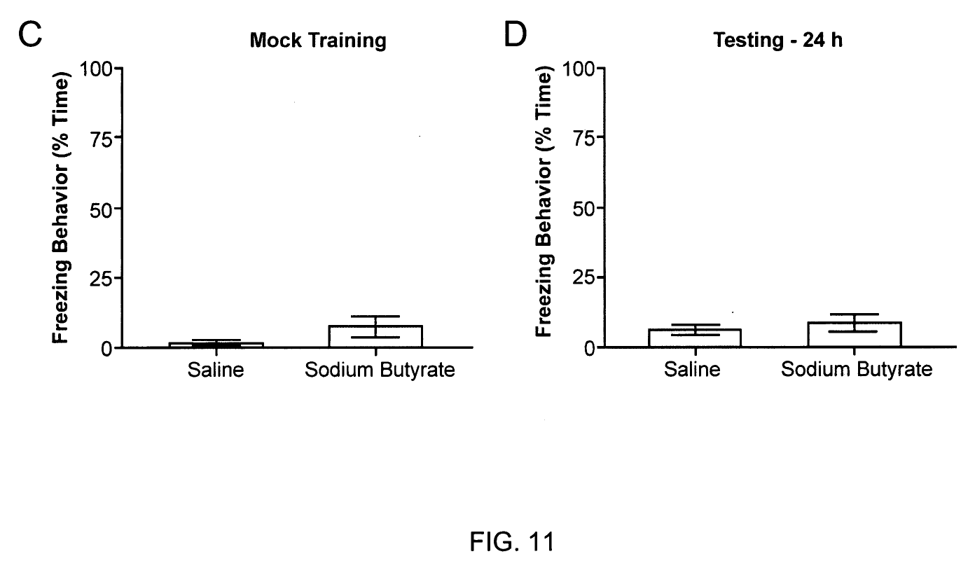

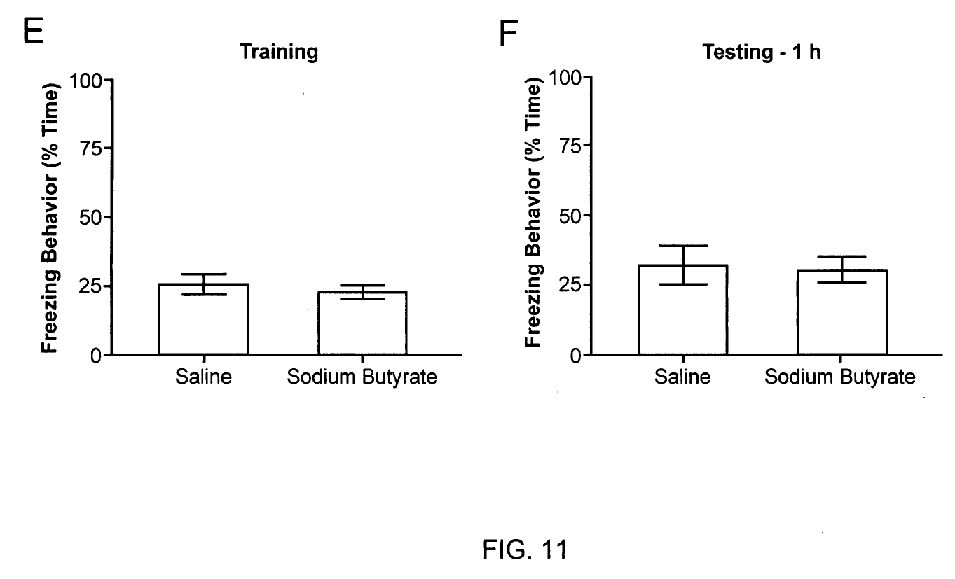

Synaptic plasticity is believed to be a mechanism involved in the formation of memory in vivo. It was observed that 2 distinct inhibitors of HDAC activity enhanced induction of LTP in vitro (FIGS. 7 and 10). Therefore, in some embodiments inhibition of HDAC activity in vivo enhances the formation of long-term contextual fear memory. To investigate this, rats were injected with NaB 1 h prior to 1-shock contextual fear conditioning. Injections of NaB had no affect on freezing behavior observed during training, indicating that NaB does not affect an animal's ability to perceive and respond to the footshock (FIG. 11A). However, when freezing behavior was assessed 24 h after training, animals injected with NaB displayed significantly more freezing behavior than animals injected with saline (FIG. 11B).

To investigate whether the injection of NaB itself was either influencing freezing behavior or inducing general malaise that was associated with presentation of the novel context, animals were exposed to the same paradigm outlined above, but no footshock was administering during the mock training session. No difference in freezing behavior was observed when animals were exposed to the training cage during the mock training session (FIG. 11C), indicating that NaB had no acute effects on freezing behavior. Animals exhibited no increase in freezing behavior upon re-exposure to the training context 24 h after the mock training trial (FIG. 11D), indicating that the footshock was required for the formation of contextual fear. Moreover, there were no significant differences in the amount of freezing behavior between saline and NaB injected animals, indicating that injection of NaB did not induce a fearful response or general malaise that was associated with the training context in the absence of the footshock.

In a final series of studies, the present inventors investigated the effect of sodium butyrate on short-term memory formation. Animals were injected with either saline or NaB and fear conditioned as above, but freezing behavior was tested 1 h after the end of the training session. As before (FIG. 11A), no significant differences were seen in freezing behavior between animals injected with either saline or NaB during training (FIG. 11E), indicating that injection of NaB had no affect on the animal's ability to perceive or respond to the training stimuli. However, injection of NaB had no effect on freezing behavior exhibited when animals were re-exposed to the training context 1 h after training (FIG. 11F). These results indicate that when administered in vivo, NaB has no effect on short-term memory formation. Therefore, the results indicate that inhibition of HDAC activity in vivo specifically enhanced the formation of long-term contextual fear memory.

### Example 9

### Enhancement of Long-Term Memory with Histone Deacetylase Inhibitor as Demonstrated with Passive Avoidance

The effect of the exemplary histone deacetylase inhibitor sodium butyrate (NaB) on the exemplary hippocampus-dependent long-term memory paradigm of passive avoidance was determined. In the passive avoidance task, animals associate a dark chamber with an aversive stimulus. In the passive avoidance task, animals are placed into the lighted side of a 2-chamber shuttle box (Med Associates). An open doorway in the middle of the shuttle box leads to a dark side. Animals are allowed to move into the dark side of the chamber, upon which time the door is closed and animals are allowed to explore the dark side for 5 sec prior to receiving a brief electric shock (2 sec, 0.5 mA). Animals are given an additional 5 sec of exploration time prior to removal from the shuttle box. Latency to enter the dark side of the shuttle box is recorded for all experimental animals during the training day. Passive avoidance memory is assessed by measuring latency to enter the dark side of the shuttle box, minus the latency to enter the dark side on the training day. Passive avoidance memory was only tested once from each animal to eliminate the effect of extinction. NaB (1.2 g/kg) was administered 45 min prior to passive avoidance training. NaB did not affect long-term passive avoidance memory when tested 1 day after training (FIG. 10). However, NaB had significant affects on passive avoidance memory when tested 7 and 14 days after training (FIG. 10, F[1,97]=5.2, p<0.05). Thus, long-term memory was significantly enhanced 7 and 14 days after training.

### Example 10

### Enhancement of Long-Term Memory with Histone Deacetylase Inhibitor as Demonstrated with Novel Object Recognition

The effect of the exemplary histone deacetylase inhibitor sodium butyrate (NaB) on the exemplary hippocampus-dependent long-term memory paradigm of novel object recognition was determined. In the novel object task, an animal's ability to discriminate between a familiar and a novel object is tested. Novel object recognition training is performed over 4 consecutive days. During each day, animals are placed individually into a small holding cage prior to introduction to the training arena (44 cm²). The first day consists of habituating animals to the training arena for 10 min. During the second and third days, the training arena has 8 small Duplo® Blocks (Lego®, 2 each: yellow, blue, red, green) placed 2 cm from the edge of the arena. During days 2 and 3, animals are simultaneously habituated to the Duplo blocks and tested for color and arena-position biases. On day 4, 2 distinct novel objects made from Duplo blocks (approx. 10×5×5 cm) are placed in opposite corners of the arena, 2 cm from the walls. Animals are allowed to explore the objects for 10 min. During each session in the arena, movement of the animal will be tracked by an automated video-based system (Limelight) and the arena and Duplo blocks are thoroughly rinsed with 10% EtOH to remove any scent cues left by subjects. During the final training session on day 4, exploration time of each object is tracked. If an animal does not explore each object for at least 90 sec, then that animal is excluded from the behavioral analyses. Novel object recognition is assessed by calculating preference indices ([Time_{Novel}−Time_{Familar}]) after exposure of animals to one familiar, and one novel object. Animals received an injection of either saline or NaB 45 min before the training session on day 4. Animals that received NaB performed significantly better than animals that received saline 1 and 7 days after training (FIG. 11, F[1,96]=13, p<0.001). A trend for enhanced performance was observed on day 14 (FIG. 11). Thus, long-term memory was significantly enhanced 1 and 7 days after training.

### Example 11

### Significance of the Present Invention

Formation of long-term memory requires the coordinated action of numerous signaling pathways to ultimately effect long-term changes in gene expression. Memory-associated signaling begins at the plasma membrane, where activation of NMDA-receptors leads to influx of Ca⁺⁺ and engagement of a variety of signaling pathways that ultimately converge on the ERK signaling cascade (Adams and Sweatt, 2002). Upon activation, ERK performs several functions relevant for establishing short- and long-term memory. In the case of long-term memory, ERK regulates the activity of a number of transcription factors including CREB and Elk-1 (Davis et al., 2000; Sananbenesi et al., 2002). These ERK-modulated transcription factors engage a complex pattern of transcriptional regulation that ultimately results in the establishment and stabilization of long-term memory.

Several recent studies suggest that chromatin structure must be altered to allow for robust and lasting changes in gene expression, especially in the nervous system (Battaglioli et al., 2002; Huang et al., 2002; Guan et al., 2002; Guan et al., 2003; Huang et al., 1999). The most efficient targets for altering the structure of chromatin are histones, the protein backbone of chromatin. Neutralizing the basic charge of histones through acetylation can lead to changes in chromatin structure, and we hypothesized that formation of long-term memory would result in significant changes in chromatin structure. In addressing this hypothesis, we observed a large change in the acetylation of histone H3 in area CA1 of the hippocampus 1 h after contextual fear conditioning. Like formation of memory itself, the change in acetylation of histone H3 required activation of NMDA-Rs, engagement of the ERK signaling cascade, and was restricted to times where critical phases of memory-associated transcription occur. This result is remarkable for several reasons. First, it indicates that changes in hippocampal chromatin structure during the early periods of long-term memory formation are widespread and dramatic in this learning paradigm. Second, this result is consistent with the emerging model that the transcription of many genes is regulated during the early stages of long-term memory formation (Levenson et al., 2004). This is not surprising, as several transcription factors have been implicated in the early stages of long-term memory formation, including CREB, C/EBP-β & δ, c-fos, erg1/zif268, Elk-1 and NFθB (Davis et al., 2000; Sananbenesi et al., 2002; Levenson et al., 2004; Bourtchuladze et al., 1994; Jones et al., 2001; Taubenfeld et al., 2001; Fleischmann et al., 2003; Meffert et al., 2003). Finally, at its most basic level the observation indicates that regulation of chromatin structure is one of the fundamental molecular mechanisms contributing to long-term memory formation.

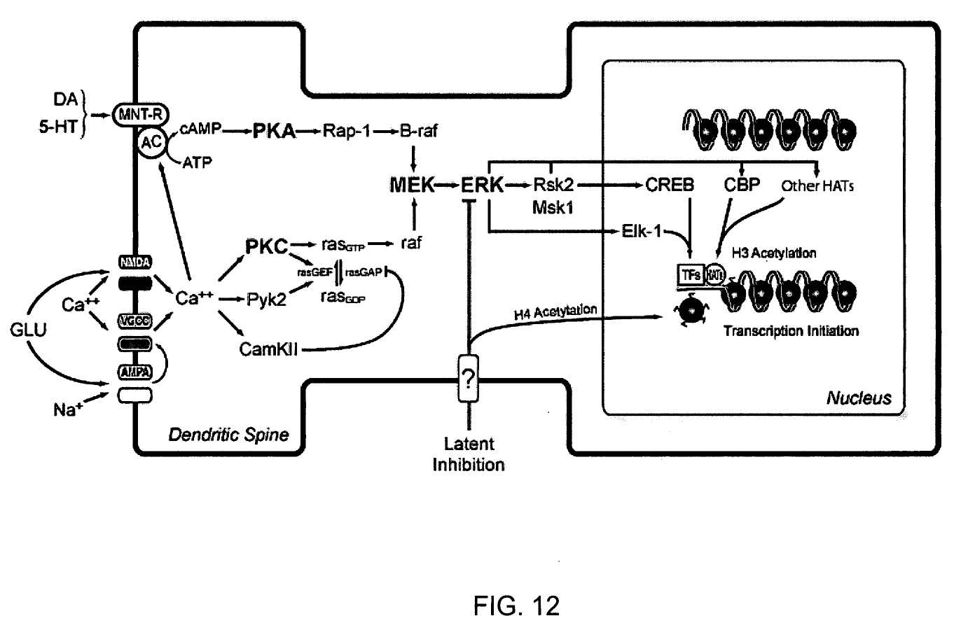

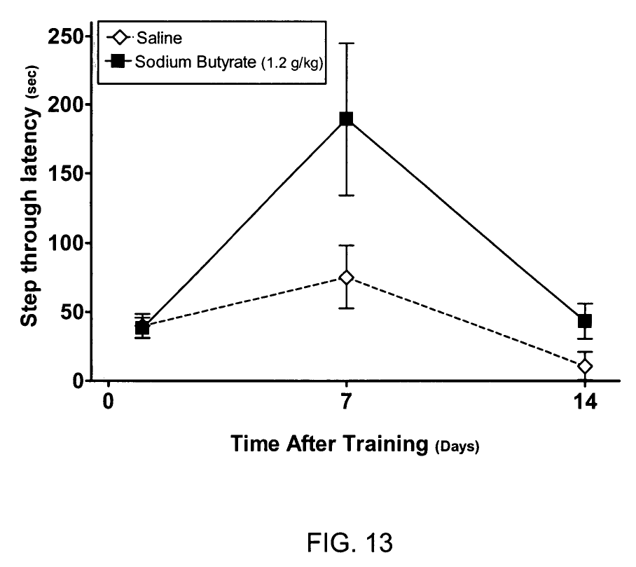

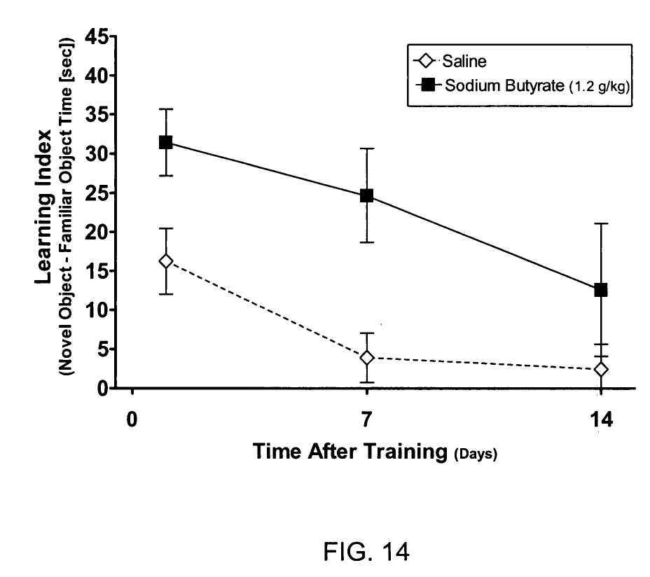

The results indicate that the ERK signaling cascade couples the activity of NMDA-Rs and two cytoplasmic signaling pathways, PKA and PKC, known to be involved in long-term memory formation (Abeliovich et al., 1993; Abel et al., 1997; Weeber et al., 2000) with the functional state of chromatin in the nucleus (FIG. 12). The exemplary model is that ERK accomplishes this through a multi-component mechanism encompassing: the direct regulation of transcription factors, such as Elk-1 (Davis et al., 2000; Sananbenesi et al., 2002); indirectly modulating the activity of transcriptional co-regulators, such as CBP (Merienne et al., 2001), and through recruiting other histone-modifying enzymes to the transcriptional complex such as histone acetyltransferases, methyltransferases and kinases (FIG. 12). Further work will be required to determine the precise molecular mechanisms through which ERK regulates histone acetylation in the hippocampus.

In addition to ERK, other signaling cascades are also likely involved in regulating chromatin structure. For example, we observed that latent inhibition training resulted in a significant increase in acetylation of histone H4, which was independent of an increase in P-ERK2 (FIG. 1D). Moreover, activation of NMDA-Rs, PKA or PKC had no effect on acetylation of histone H4 in vitro (FIG. 6), indicating that the signaling pathways governing acetylation of histone H4 are very different from the signaling pathways regulating acetylation of histone H3. Given that latent inhibition training prevents formation of contextual fear memory, in some embodiments the resulting changes in transcription mediated by histone H4 act to antagonize histone H3-mediated changes in transcription. Some evidence indicates that signaling via p38 MAP kinase is independent of and acts to antagonize ERK signaling and vice versa (Guan et al., 2003; Blum et al., 1999; Murray and O'Connor, 2003). Therefore, in one embodiment the regulation of histone H4 by latent inhibition is due to p38 MAP kinase signaling.

The present inventors observed that inhibition of histone deacetylases, enzymes that promote the formation of transcriptionally silent heterochromatin, augmented the formation of LTP in vitro and long-term memory in vivo. How could an inhibitor of histone deacetylase enhance synaptic plasticity and long-term memory formation without having any affect on basal properties of synaptic transmission or animal behavior? Previous studies indicate that inhibition of histone deacetylase only affects the expression of a restricted set of genes (Van Lint et al., 1996). Moreover, a contemporary model of long-term memory formation posits that under normal conditions, transcriptional repressors of memory formation dominate transcriptional activators, so that under normal conditions long-term synaptic enhancement and memory formation are prevented (Abel and Kandel, 1998). Transcriptional repressors recruit histone deacetylases to DNA, resulting in the formation of transcriptionally silent heterochromatin, whereas activators of transcription recruit histone acetyltransferases, which increase histone acetylation and result in the formation of transcriptionally competent euchromatin. Therefore, in some embodiments histone deacetylase inhibitors are acting to decrease the efficacy of memory repressors, essentially priming the transcriptional machinery. This embodiment is supported by the observation that inhibiting transcription blocks the effect of HDAC inhibitors on LTP. The transcriptional priming results in enhanced induction of LTP and long-term memory formation. Augmentation of long-term forms of synaptic plasticity and long-term memory formation has also been seen by disrupting the inhibitory forms of CREB, a critical transcription factor in the formation of long-term memory (yin et al., 1995; Yin et al., 1994; Bartsch et al., 1998; Bartsch et al., 1995). Therefore, in some embodiments HDAC inhibitors represent a viable route for the treatment of cognitive impairment, or for enhancing memory in people with normal cognitive ability.

Modification of histones, either through acetylation, phosphorylation or methylation, is a biochemical memory that can persist beyond the initial signaling event in many cell types (Paro, 1995). To date, these sorts of changes have largely been identified as a mechanism for triggering cellular differentiation that persists indefinitely (Orlando, 2003; Muller and Leutz, 2001). An interesting current hypothesis in the field of developmental biology is that changes in the acetylation of histones persist as a cellular form of memory of previous cytoplasmic signaling events (Paro, 1995; Jenuwein and Allis, 2001). For example, the transcription factor REST is responsible for inhibiting the expression of a set of genes that would impart a neuronal phenotype to a cell. Once induced to differentiate into a non-neuronal cell, REST recruits a number of proteins including co-repressors and histone deacetylases to effect a permanent change in chromatin structure around neuronal genes (Battaglioli et al., 2002; Lunyak et al., 2002). Therefore, a brief signaling event that induced differentiation resulted in a lasting change in gene expression effected through changes in chromatin structure. In fact, epigenetic change in chromatin structure is one of the most pervasive forms of cellular memory—“remembered” long-term changes in gene expression occur in cells as dissimilar as bacteria and neurons (Casadesus and D'Ari, 2002). Thus, in some embodiments changes in acetylation of histones not only represents a mechanism for acute changes in transcription of genes relevant for memory formation in neurons, but rather is a conserved mechanism for cellular memory in general.

The present invention reveals a new molecular correlate of long-term memory formation: alterations in chromatin structure. Changes in chromatin structure were observed that accompanied long-term memory formation and activation of second messenger signaling pathways that are known to mediate induction of long-term synaptic potentiation. Interestingly, by circumventing the normal signaling pathways involved in the formation of memory and mimicking these changes in chromatin structure by pharmacologic means, we actually enhanced the capacity for Schaffer-collateral synapses to express LTP in vitro and enhanced the formation of long-term memory in vivo. These results indicate the interesting embodiment wherein chromatin structure itself represents a “memory”, allowing for temporal integration of spaced signals or metaplasticity of synapses.

### REFERENCES

All publications mentioned herein are incorporated herein by reference for the purpose of describing and disclosing, for example, the methods and compositions that are described in the publications that might be used in connection with the presently described invention. The publications discussed above and throughout the text are provided solely for their disclosure prior to the filing date of the present application. Nothing herein is to be construed as an admission that the inventors are not entitled to antedate such disclosure by virtue of prior invention. All patents, patent applications, and publications mentioned herein are hereby incorporated herein by reference.

### Patents and Patent Applications

U.S. 2004/0077591

### Publications

Abel, T., and Kandel, E. (1998) Brain Res Brain Res Rev 26, 360-378.
Abel, T., Nguyen, P. V., Barad, M., Deuel, T. A., Kandel, E. R., and Bourtchouladze, R. (1997) Cell 88, 615-626
Abeliovich, A., Paylor, R., Chen, C., Kim, J. J., Wehner, J. M., and Tonegawa, S. (1993) Cell 75, 1263-1271
Adams, J. P., and Sweatt, J. D. (2002) Annu Rev Pharmacol Toxicol 42, 135-163
Angus-Hill, M. L., Dutnall, R. N., Tafrov, S. T., Sternglanz, R., and Ramakrishnan, V. (1999) J Mol Biol 294, 1311-1325
Atkins, C. M., Selcher, J. C., Petraitis, J. J., Trzaskos, J. M., and Sweatt, J. D. (1998) Nat Neurosci 1, 602-609
Bartsch, D., Casadio, A., Karl, K. A., Serodio, P., and Kandel, E. R. (1998) Cell 95, 211-223
Bartsch, D., Ghirardi, M., Skehel, P. A., Karl, K. A., Herder, S. P., Chen, M., Bailey, C. H., and Kandel, E. R. (1995) Cell 83, 979-992
Battaglioli, E., Andres, M. E., Rose, D. W., Chenoweth, J. G., Rosenfeld, M. G., Anderson, M. E., and Mandel, G. (2002) J Biol Chem 277, 41038-41045
Blum, S., Moore, A. N., Adams, F., and Dash, P. K. (1999) J Neurosci 19, 3535-3544
Bourtchuladze, R., Frenguelli, B., Blendy, J., Cioffi, D., Schutz, G., and Silva, A. J. (1994) Cell 79, 59-68
Calderone, A., Jover, T., Noh, K. M., Tanaka, H., Yokota, H., Lin, Y., Grooms, S. Y., Regis, R., Bennett, M. V., and Zukin, R. S. (2003) J Neurosci 23, 2112-2121
Casadesus, J., and D'Ari, R. (2002) Bioessays 24, 512-518
Cheung, P., Tanner, K. G., Cheung, W. L., Sassone-Corsi, P., Denu, J. M., and Allis, C. D. (2000) Mol Cell 5, 905-915
Crosio, C., Cermakian, N., Allis, C. D., and Sassone-Corsi, P. (2000) Nat Neurosci 3, 1241-1247
Crosio, C., Heitz, E., Allis, C. D., Borrelli, E., and Sassone-Corsi, P. (2003) J Cell Sci 116, 4905-4914
Davis, S., Vanhoutte, P., Pages, C., Caboche, J., and Laroche, S. (2000) J Neurosci 20, 4563-4572
Dingledine, R. (1983) Fed Proc 42, 2881-2885
English, J. D., and Sweatt, J. D. (1997) J Biol Chem 272, 19103-19106
Fanselow, M. S., Kim, J. J., Yipp, J., and De Oca, B. (1994) Behav Neurosci 108, 235-240
Fleischmann, A., Hvalby, O., Jensen, V., Strekalova, T., Zacher, C., Layer, L. E., Kvello, A., Reschke, M., Spanagel, R., Sprengel, R., Wagner, E. F., and Gass, P. (2003) J Neurosci 23, 9116-9122
Freedman, L. (1999) Cell 97, 5-8
Frey, U., Frey, S., Schollmeier, F., and Krug, M. (1996) J Physiol 490 (Pt 3), 703-711
Frey, U., Krug, M., Brodemann, R., Reymann, K., and Matthies, H. (1989) Neurosci Lett 97, 135-139
Frey, U., Krug, M., Reymann, K. G., and Matthies, H. (1988) Brain Res 452, 57-65
Guan, Z., Giustetto, M., Lomvardas, S., Kim, J. H., Miniaci, M. C., Schwartz, J. H., Thanos, D., and Kandel, E. R. (2002) Cell 111, 483-493
Guan, Z., Kim, J. H., Lomvardas, S., Holick, K., Xu, S., Kandel, E. R., and Schwartz, J. H. (2003) J Neurosci 23, 7317-7325
Harris, E. W., Ganong, A. H., and Cotman, C. W. (1984) Brain Res 323, 132-137
Huang, Y., Doherty, J. J., and Dingledine, R. (2002) J Neurosci 22, 8422-8428.
Huang, Y., Myers, S. J., and Dingledine, R. (1999) Nat Neurosci 2, 867-872.
Igaz, L. M., Vianna, M. R., Medina, J. H., and Izquierdo, I. (2002) J Neurosci 22, 6781-6789
Impey, S., Smith, D. M., Obrietan, K., Donahue, R., Wade, C., and Storm, D. R. (1998) Nat Neurosci 1, 595-601
Jenuwein, T., and Allis, C. D. (2001) Science 293, 1074-1080
Jones, M. W., Errington, M. L., French, P. J., Fine, A., Bliss, T. V., Garel, S., Charnay, P., Bozon, B., Laroche, S., and Davis, S. (2001) Nat Neurosci 4, 289-296
Levenson, J. M., Choi, S., Lee, S. Y., Cao, Y. A., Ahn, H. J., Worley, K. C., Pizzi, M., Liou, H. C., and Sweatt, J. D. (2004) J Neurosci 24, 3933-3943
Lubow, R. E. (1973) Psychol Bull 79, 398-407
Lunyak, V. V., Burgess, R., Prefontaine, G. G., Nelson, C., Sze, S. H., Chenoweth, J., Schwartz, P., Pevzner, P. A., Glass, C., Mandel, G., and Rosenfeld, M. G. (2002) Science 298, 1747-1752
Meffert, M. K., Chang, J. M., Wiltgen, B. J., Fanselow, M. S., and Baltimore, D. (2003) Nat Neurosci 6, 1072-1078
Merienne, K., Pannetier, S., Harel-Bellan, A., and Sassone-Corsi, P. (2001) Mol Cell Biol 21, 7089-7096
Muller, C., and Leutz, A. (2001) Curr Opin Genet Dev 11, 167-174
Murray, H. J., and O'Connor, J. J. (2003) Neuropharmacology 44, 374-380
Nguyen, P. V., Abel, T., and Kandel, E. R. (1994) Science 265, 1104-1107
Orlando, V. (2003) Cell 112, 599-606
Orphanides, G., and Reinberg, D. (2000) Nature 407, 471-475
Paro, R. (1995) Trends Genet 11, 295-297
Sananbenesi, F., Fischer, A., Schrick, C., Spiess, J., and Radulovic, J. (2002) Mol Cell Neurosci 21, 463-476
Selcher, J. C., Atkins, C. M., Trzaskos, J. M., Paylor, R., and Sweatt, J. D. (1999) Learn Mem 6, 478-490
Stanton, P. K., and Sarvey, J. M. (1984) J Neurosci 4, 3080-3088
Taubenfeld, S. M., Wiig, K. A., Monti, B., Dolan, B., Pollonini, G., and Alberini, C. M. (2001) J Neurosci 21, 84-91
Turner, B. M. (2002) Cell 111, 285-291
Van Lint, C., Emiliani, S., and Verdin, E. (1996) Gene Expr 5, 245-253
Varga-Weisz, P., and Becker, P. (1998) Curr Opin Cell Biol 10, 346-353
Weeber, E. J., Atkins, C. M., Selcher, J. C., Varga, A. W., Mirnikjoo, B., Paylor, R., Leitges, M., and Sweatt, J. D. (2000) J Neurosci 20, 5906-5914
Yin, J. C., Del Vecchio, M., Zhou, H., and Tully, T. (1995) Cell 81, 107-115
Yin, J. C., Wallach, J. S., Del Vecchio, M., Wilder, E. L., Zhou, H., Quinn, W. G., and Tully, T. (1994) Cell 79, 49-58

Although the present invention and its advantages have been described in detail, it should be understood that various changes, substitutions and alterations can be made herein without departing from the invention as defined by the appended claims. Moreover, the scope of the present application is not intended to be limited to the particular embodiments of the manufacture, composition of matter, means, methods and steps described in the specification. As one will readily appreciate from the disclosure, processes, machines, manufacture, compositions of matter, means, methods, or steps, presently existing or later to be developed that perform substantially the same function or achieve substantially the same result as the corresponding embodiments described herein may be utilized. Accordingly, the appended claims are intended to include within their scope such processes, machines, manufacture, compositions of matter, means, methods, or steps.

What is claimed is:

1. A method of enhancing cognition in an individual, comprising the step of delivering to the individual an effective amount of a histone acetylation regulator.

2. The method of claim 1, wherein the individual comprises a medical condition selected from the group consisting of mild cognitive impairment (MCI), aging-related memory impairment, non-syndromic mental retardation, Rett syndrome, fragile X mental retardation, Down syndrome, attention deficit disorder, developmental delay, Angelman syndrome, or cognitive enhancement in low-IQ individuals.

3. The method of claim 1, wherein the histone acetylation regulator comprises a histone acetyltransferase activator or activity enhancer.

4. The method of claim 1, wherein the histone acetylation regulator comprises a histone deacetylase inhibitor.

5. The method of claim 4, wherein the histone deacetylase inhibitor comprises trichostatin A, trichostatin B, trichostatin C, trapoxin A, trapoxin B, chlamydocin, sodium butyrate, sodium phenylbutyrate, MS-27-275, scriptaid, FR901228, depudecin, oxamflatin, pyroxamide, apicidin B, apicidin C, Helminthsporium carbonum toxin, 2-amino-8-oxo-9,10-epoxy-decanoyl, 3-(4-aroyl-1H-pyrrol-2-yl)-N-hydroxy-2-propenamide, suberoylanilide hydroxamic acid, FK228, m-carboxycinnamic acid bis-hydroxamide, or a mixture thereof.

6. The method of claim 1, further defined as enhancing memory in the individual.

7. The method of claim 6, further defined as enhancing long-term memory in the individual.

8. The method of claim 1, wherein said individual comprises substantially normal memory faculty.

9. The method of claim 1, wherein the individual comprises substantially sub-normal memory faculty.

10. The method of claim 9, wherein the sub-normal memory faculty results from a pathogenic condition.

11. The method of claim 10, wherein the pathogenic condition comprises a mental retardation syndrome, mild cognitive impairment (MCI), attention deficit disorder, developmental delay, aging-related memory impairment, cognitive enhancement in low-IQ individuals, non-syndromic mental retardation, bipolar disorder, head trauma, seizures, alcoholism, stroke, transient ischemic attack (TIA), transient global amnesia, electroconvulsive therapy, brain mass, infection, depression, AIDS-related cognitive impairment, or a combination thereof.

12. The method of claim 11, wherein the mental retardation syndrome is Rett syndrome, fragile X mental retardation, Down syndrome, Angelman syndrome.

13. The method of claim 9, wherein the sub-normal memory faculty results from a non-pathogenic condition.

14. The method of claim 13, wherein the non-pathogenic condition comprises anesthesia, an illicit/illegal drug, temporal lobe brain surgery, or a combination thereof.

15. The method of claim 14, wherein the anesthesia comprises halothane, isoflurane, fentanyl, or a mixture thereof.

16. The method of claim 14, wherein the illicit/illegal drug comprises barbiturates, benzodiazepines, marijuana, cocaine, heroin, methamphetamine, inhalants, Ecstasy, GHB, ketamine or a mixture thereof.

17. The method of claim 13, wherein the non-pathogenic condition comprises normal age-related impairment.

18. The method of claim 17, wherein the normal age-related impairment comprises mild cognitive impairment (MCI).

19. The method of claim 1, further defined as reducing the activity of a nuclear memory repressor.

20. The method of claim 1, wherein said method does not substantially affect basal synaptic transmission, short-term plasticity, or both.

21. A method of improving memory in an individual having a disease associated therewith, comprising the step of delivering to the individual an effective amount of a histone acetylation regulator, wherein the disease comprises a mental retardation syndrome, mild cognitive impairment (MCI), attention deficit disorder, developmental delay, aging-related memory impairment, cognitive enhancement in low-IQ individuals, non-syndromic mental retardation, bipolar disorder, head trauma, seizures, alcoholism, stroke, transient ischemic attack (TIA), transient global amnesia, electroconvulsive therapy, brain mass, infection, depression, AIDS-related cognitive impairment, or a combination thereof.

22. A method of treating at least one cognition-associated symptom of an individual with mental retardation, comprising the step of delivering to the individual a therapeutically effective amount of a histone acetylation regulator.

23. The method of claim 22, wherein the cognition-associated symptom comprises learning disability, substantially sub-normal memory faculty, or a combination thereof.

24. The method of claim 22, wherein the histone acetylation regulator is a histone deacetylase inhibitor.
